(19) United States
(12) Patent Application Publication
(10) Pub. No.: US 2024/0181013 A1
(43) Pub. Date: Jun. 6, 2024

(54) POEGMA COPOLYMER CONJUGATES AND METHODS OF TREATING DISEASES

(71) Applicant: Duke University, Durham, NC (US)

(72) Inventors: Ashutosh Chilkoti, Durham, NC (US); Imran Ozer, Durham, NC (US)

(21) Appl. No.: 18/553,135

(22) PCT Filed: Apr. 1, 2022
(86) PCT No.: PCT/US2022/023158

§ 371 Date: Sep. 28, 2023

Related U.S. Application Data
(60) Provisional application No. 63/169,541, filed on Apr. 1, 2021.

(51) Int. Cl. A61K 38/22; A61K 47/60; A61P 3/10; C07K 14/575

(52) U.S. Cl. CPC A61K 38/2278; A61K 47/60; A61P 3/10; C07K 14/57563

(57) ABSTRACT

Disclosed are POEGMA copolymer conjugates that have phase transition behavior and a reduced or eliminated host-immune response. An example conjugate includes a biologically active agent and a copolymer of POEGMA conjugated to the biologically active agent where the conjugate has a transition temperature. The POEGMA copolymer conjugates can form drug releasing depots. which can be useful in methods of treating diseases.

### CROSS-REFERENCE TO RELATED APPLICATIONS

This application claims priority to U.S. Provisional Patent Application No. 63/169,541 filed on Apr. 1, 2021, which is incorporated fully herein by reference.

### TECHNICAL FIELD

This disclosure relates to poly[oligo(ethylene glycol) ether methacrylate] (POEGMA) copolymer conjugates and their use in treating diseases

### BACKGROUND

Biologics are potent, highly specific, well-tolerated, and have become an important class of drugs. Despite their promise, most of them have a short half-life due to their small size and poor stability in circulation, leading to rapid elimination. Rapid clearance of these drugs necessitates frequent injections, resulting in sharp changes in the circulating drug concentration, high treatment cost, and suboptimal patient compliance. One of the most common approaches to overcome these challenges is their covalent attachment to polyethylene glycol (PEG), termed PEGylation.

PEGylated drug conjugates have a much longer plasma half-life than the native drug due to their larger size, improved solubility, and stability. Unfortunately, PEGylation has significant limitations. First, PEG was initially believed to be non-immunogenic. However, PEGylated therapeutics induce PEG antibodies upon treatment. Pre-existing PEG antibodies have also been reported in up to 70% of the population who are naïve to PEGylated therapeutics, possibly due to chronic exposure to PEG in consumer products. Both induced and pre-existing PEG antibodies can cause a severe allergic reaction and forced accelerated clearance in patients, reducing the drugs' clinical efficacy. These setbacks have collectively led to the early termination of PEGylated drug candidates' clinical trials and the withdrawal of several PEGylated therapeutics from the market. Second, attempts to improve the pharmacokinetics (PK) of PEG have focused on synthesizing branched and star-shaped PEGs. However, these architectures have a modest effect on PK and have antigenic and immunogenic profiles similar to linear PEG. Third, PEG forms vacuoles in major organs due to its non-biodegradable structure and clearance by the reticuloendothelial cells. Accordingly, new polymer architectures would be useful in overcoming these drawbacks of PEG.

SUMMARY

In one aspect, disclosed are conjugates including a biologically active agent; and a copolymer of poly[oligo(ethylene glycol) ether methacrylate] (POEGMA) conjugated to the biologically active agent, the copolymer of POEGMA comprising recurring units of formula (I)

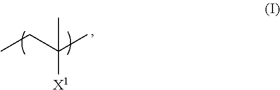

wherein
X¹ is of formula (II)

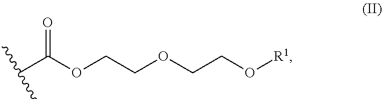

or formula (III)

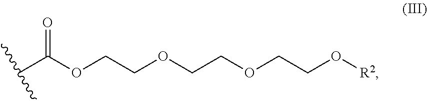

wherein R¹ and R² are each independently hydrogen, alkyl, ester, C₁-C₄ alkylenyl-NH₂, amide, carboxyl, or C₁-C₄ alkylenyl-OH, wherein the copolymer of POEGMA comprises about 1 molar % to about 99 molar % of recurring units with formula (II), about 1 molar % to about 99 molar % of recurring units with formula (III), and a weight average molecular weight of about 2 kDa to about 500 kDa, and wherein the conjugate has a transition temperature (Tₜ) of about 25° C. to about 37° C. at a concentration of about 1 uM to about 1 M.

In another aspect, disclosed are compositions including a plurality of conjugates as disclosed herein, wherein the plurality of conjugates self-assemble into an aggregate above the Tₜ of the conjugate.

In another aspect, disclosed are methods of treating a disease in a subject in need thereof, the method comprising administering to the subject an effective amount of the composition as disclosed herein, wherein the composition does not induce a histopathological change in the subject.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-D show POEGMAs at varying molecular weight (M_{w}) and monomer composition phase-transition near body temperature in a concentration- and M_{w}-dependent manner FIG. 1A: gel permeation chromatography (GPC) chromatogram of EG₂₆₆% copolymers at varied M_{w}. FIG. 1B: The optical density of POEGMAs with constant degree of polymerization (DP) but varying monomer composition was monitored as temperature increased (solid line) and decreased (dashed line) to demonstrate the reversibility of phase behavior. Data were shown for POEGMAs at DP 200 and 25 μM. FIG. 1C: At varying concentrations, the optical density of POEGMAs demonstrates concentration-dependence of transition temperature (Tₜ). Data were shown for POEGMAs at DP 300 in phosphate-buffered saline (PBS). FIG. 1D: The optical density of POEGMAs with varying DP and monomer composition demonstrate Tₜ's DP-dependence. Data were shown for POEGMAs at 25 μM in PBS.

FIG. 2A-L show an exendin-POEGMA conjugate that maximizes fed blood glucose control in diabetic mice. FIG. 2A: The optical density of exendin-POEGMA conjugates with similar M_{w}. but varied Tₜ as temperature increased (solid line) and decreased (dashed line), demonstrating the reversibility of phase behavior at 25 μM and FIG. 2B: at varying concentrations to demonstrate inverse concentration-dependence of Tₜ. Circled data shows Tₜ at the injection concentration of 500 μM. FIG. 2C: In vitro activity of the conjugates (n=6). FIG. 2D: Blood glucose normalized to t=0, FIG. 2E: area under the curve (AUC) of blood glucose, and FIG. 2F: percent weight change relative to weight at t=0 after treating eleven-week-old diet-induced obese (DIO) C₅₇BL/6J mice (n=6) with a single s.c. injection of the treatments. FIG. 2G: The optical density of exendin-POEGMA conjugates with the same Tₜ but varied M_{w} as temperature increased, demonstrating phase behavior at 25 μM and FIG. 2H: at varying concentrations to demonstrate inverse concentration-dependence of Tₜ. FIG. 21: In vitro activity of the conjugates (n=6). FIG. 2J: Blood glucose normalized to t=0, FIG. 2K: AUC of blood glucose, and FIG. 2L: percent weight change relative to weight at t=0 after treating DIO C₅₇BL/6J mice (n=6) with a single s.c. injection of the treatments. Blood glucose was analyzed using a t-test between a treatment group and PBS. * Last time point that blood glucose was significantly lower than PBS. Glucose exposure was analyzed using one-way ANOVA, followed by Sidak's multiple comparison test. ^{#}The conjugate with the lowest glucose exposure. Data showed the mean±standard error of the mean (SEM) and considered statistically significant when p<0.05.

FIG. 3A-L show that exendin-POEGMAₒₚₜ outperforms its soluble POEGMA and PEG counterparts and a clinical sustained-release exendin formulation, Bydureon, in fed blood glucose and glycemic control. The optical density of treatments FIG. 3A: as temperature increased and decreased, demonstrating reversible phase behavior for Ex-POEGMAopt and Ex-POEGMAₛₒₗ at 25 μM and FIG. 3B: at varying concentrations to demonstrate inverse concentration-dependence of Tₜ. Circled data show Tₜ at the injection concentration of 500 μM. FIG. 3C: In vitro activity of the conjugates (n=6). FIG. 3D: Blood glucose normalized to t=0, FIG. 3E: AUC of blood glucose, FIG. 3F: and percent weight change relative to weight at t=0 after treating eleven-week-old DIO mice (n=6) with a single s.c. injection of the treatments. FIG. 3G: Blood glucose normalized to t=0, FIG. 3H: AUC of blood glucose, FIG. 3F: and percent weight change relative to weight at t=0 after treating six-week-old db/db mice (n=6) with a single s.c. injection of the treatments. An intraperitoneal glucose tolerance test (IPGTT) was performed on FIG. 3J. day 1 and FIG. 3K: day 5 post-injection of treatments into db/db mice (n=5), blood glucose was monitored, and FIG. 3L: AUC of blood glucose was quantified. Blood glucose was analyzed using a t-test between a treatment group and PBS. *Last time point that blood glucose was significantly lower than PBS. Glucose exposure was analyzed using one-way ANOVA, followed by Sidak's multiple comparison test. ^{#}The treatment with the lowest glucose exposure. Data showed the mean±SEM and considered statistically significant when p<0.05.

FIG. 4A-E show pharmacokinetics of exendin-POEGMAₒₚₜ. Fluorescently labeled FIG. 4A: exendin and FIG. 4B: Ex-PEG_{Mw}, Ex-PEG_{Rh}, and Ex-POEGMAₒₚₜ were s.c. administrated into naïve male DIO C₅₇BL/6J mice (n =4) at 1000 nmol kg⁻¹, followed by blood collection at specified time points for 168 hours for calculation of drug concentration. The treatments were administrated four more times to immunize the mice and induce anti-drug antibodies. At the last injection, concentrations of fluorescently labeled FIG. 4C: exendin, FIG. 4D: Ex-PEG_{Rh}, and (E) Ex-POEGMAₒₚₜ were tracked (dotted lines). P values were shown for AUC comparison between naïve (solid line) and immunized mice for each treatment. Fluorophore concentration was 45 nmol kg¹. Data represent the mean and standard error of the mean (SEM).

FIG. 5A-C show long-term efficacy of Ex-POEGMAₒₚₜ FIG. SA: Blood glucose normalized to t-0 and FIG. 5B: area under the curve (AUC) of blood glucose were monitored after treating six-week-old B6.BKS(D)-Lepr^{db}/J mice (n=5) with weekly subcutaneous injections of exendin, Ex-PEG_{Rh}, Ex-POEGMAₒₚₜ Bydureon, or PBS for eight weeks. FIG. 5C: Percent glycated hemoglobin (Hb1Ac %) level. Data represent the mean and SEM. Glucose exposure was analyzed by one-way repeated-measures ANOVA, followed by Sidak's multiple comparison test. HbA1c % was analyzed by two-way repeated-measures ANOVA, followed by Tukey's multiple comparison test. Data were considered statistically significant when p<0.05.

FIG. 6A-D show that Ex-POEGMAopt does not induce anti-POEGMA antibodies. FIG. 6A: Timeline of PBS, exendin, exendin-PEG, or exendin-POEGMAₒₚₜ administration into DIO C₅₇BL/6J mice (n=10) and blood collection. IgM subtype anti-drug antibody (ADA) response on FIG. 6B: Day 10, FIG. 6C: Day 27, and FIG. 6D: Day 44. Blood samples were collected from mice repeatedly treated with PBS, exendin, Ex-PEG_{Mw}, or Ex-POEGMAₒₚₜ. ADA response was analyzed using a Luminex multiplexed immunoassay platform. Exendin-PEG- and exendin-POEGMA-coupled beads were used to determine ADAs induced towards the entire conjugate (i.e., anti-exendin and anti-polymer (PEG or POEGMA)). OVA-PEG- and OVA-POEGMA-coupled beads were used to determine ADAs induced towards PEG or POEGMA, respectively. The OVA-coupled bead was used as a negative control for cross-reactivity towards OVA. Data represent the mean ADA response induced in each mouse and the SEM. Data were analyzed by two-way repeated-measures ANOVA, followed by Tukey's multiple comparison test. Data were considered statistically significant when p<0.05.

### DETAILED DESCRIPTION

Disclosed herein are biologically active agents conjugated to copolymers of POEGMA that can form gel-like injectable depots. Depots of these POEGMA copolymer conjugates can achieve sustained-release from the depot into the bloodstream while preserving the conjugate's lack of reactivity towards induced PEG antibodies. POEGMA-conjugate depots were found to maximize the therapeutic benefit of the conjugated biologically active agent. In addition, POEGMA conjugates were non-immunoreactive and did not induce vacuolization. Solving the aforementioned problems of PEG by creating injectable POEGMA conjugates that can form a drug depot under the skin and provide sustained efficacy offers a promising alternative to PEG-based systems.

### 1. Definitions

Unless otherwise defined, all technical and scientific terms used herein have the same meaning as commonly understood by one of ordinary skill in the art. In case of conflict, the present document, including definitions, will control. Example methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising.” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alkyl” refers to a straight or branched, saturated hydrocarbon chain containing from 1 to 10 carbon atoms. The term “C₁-C₃ alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkylenyl” refers to a divalent group derived from a straight or branched chain hydrocarbon of 1 to 20 carbon atoms, for example, of 1 to 4 carbon atoms. Representative examples of alkylenyl include, but are not limited to, —CH₂—CH₂—, —CH₂—CH₂—CH₂—, —CH₂—CH₂—CH₂—CH₂—, and —CH₂—CH₂—CH₂—CH₂—CH₂—.

The term “amide” refers to the group —C(O)NR wherein R is selected from the group consisting of hydrogen, alkyl, alkenyl, and alkynyl, any of which may be optionally substituted, e.g., with one or more substituents.

The term “antigen” refers to a molecule capable of being bound by an antibody or a T cell receptor. The term “antigen” also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, polymers, polymer conjugates, and combinations thereof. Antigens may also be mixtures of several individual antigens.

The term “antigenicity” refers to the ability of an antigen to specifically bind to a T cell receptor or antibody and includes the reactivity of an antigen toward pre-existing antibodies in a subject.

The term “biologically active agent” refers to a substance that can act on a cell, virus, tissue, organ, organism, or the like, to create a change in the functioning of the cell, virus, tissue, organ, or organism. Examples of a biologically active agent include, but are not limited to, small molecule drugs, lipids, proteins, peptides, and nucleic acids. A biologically active agent is capable of treating and/or ameliorating a condition or disease, or one or more symptoms thereof, in a subject. Biologically active agents of the present disclosure also include prodrug forms of the agent.

The term “carboxyl” refers to the group —C(═O)OR, wherein R is selected from the group consisting of hydrogen, alkyl, alkenyl, and alkynyl, any of which may be optionally substituted, e.g., with one or more substituents.

The term “effective amount” or “therapeutically effective amount” refers to an amount sufficient to effect beneficial or desirable biological and/or clinical results.

The term “ester” refers to the group —C(O)OR wherein R is selected from the group consisting of hydrogen, alkyl, alkenyl, and alkynyl, any of which may be optionally substituted, e.g., with one or more substituents.

The term “hydroxyl” or “hydroxy” refers to an —OH group.

The term “immunogenicity” refers to the ability of any antigen to induce an immune response and includes the intrinsic ability of an antigen to generate antibodies in a subject. As used herein, the terms “antigenicity” and “immunogenicity” refer to different aspects of the immune system and are not interchangeable.

The term “subject” includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

The term “transition temperature” or “Tₜ” refers to the temperature at which the conjugate (or copolymer of POEGMA) changes from one state to another, for example, soluble to insoluble. For example, below the Tₜ, the conjugate may be highly soluble. Upon heating above the transition temperature, for example, the conjugate may aggregate, forming a separate phase. The Tₜ can also be defined as the inflection point of temperature versus the optical density curve and calculated as the maximum of the first derivative using, e.g., GraphPad Prism 8.0 software.

The term “treatment” or “treating” refers to protection of a subject from a disease, such as preventing, suppressing, repressing, ameliorating, or completely eliminating the disease. Preventing the disease involves administering a composition of the present disclosure to a subject prior to onset of the disease. Suppressing the disease involves administering a composition of the present disclosure to a subject after induction of the disease but before its clinical appearance. Repressing or ameliorating the disease involves administering a composition of the present disclosure to a subject after clinical appearance of the disease.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Conjugates

Disclosed herein are conjugates that include a biologically active agent and a copolymer of POEGMA conjugated to the biologically active agent. The conjugate has a reduced or eliminated immune response compared to a PEG-biologically active agent counterpart and has phase transition behavior. The disclosed conjugates can phase transition into drug delivering aggregates (e.g., depots) and deliver a sustained release of the conjugate without the immune and tissue (e.g., vacuolization) complications that face PEG-based systems.

It has been found that replacing the PEG of a PEG-biologically active agent conjugate with a copolymer of POEGMA can reduce or eliminate the immune response directed to the POEGMA-biologically active agent conjugate compared to the PEG-biologically active agent conjugate. The reduced or eliminated immune response can include a reduced or eliminated antigenicity, a reduced or eliminated immunogenicity, or both of the conjugate. Accordingly, the disclosed conjugate can have beneficial interactions with a subject's immune system.

The beneficial immune interactions of the conjugate can also be seen in that the conjugate may not induce an anti-POEGMA antibody response. An anti-POEGMA antibody response can include inducing IgG class antibodies, inducing IgM class antibodies, inducing a IgM response that lasts longer than 10 days, or a combination thereof. Accordingly, in some embodiments, the conjugate does not induce anti-POEGMA IgG class antibodies, induce anti-POEGMA IgM class antibodies, and/or induce an anti-POEGMA IgM response that lasts longer than 10 days. In addition, in some embodiments, the conjugate is not reactive with pre-existing anti-PEG antibodies in a subject.

With respect to the PEG-biologically active agent conjugate, this molecule can be considered a control as to what the disclosed conjugate is compared to when assessing reducing or eliminating antigenicity, immunogenicity, or both. The control can be of similar molecular weight, similar physical dimensions, or both. The control can also be branched or linear, as long as it has more than the disclosed number of consecutive ethylene glycol monomers in tandem. For example, a suitable control PEG can include linear or branched PEG having more than 3 consecutive ethylene glycol monomers in tandem.

The disclosed conjugates also have phase transition behavior. Phase transition refers to the aggregation of the conjugate(s), which may occur sharply, and in some instances, reversibly at or above a Tₜ of the conjugate. Below the Tₜ, for example, the conjugate may be highly soluble. Upon heating above the transition temperature, for example, the conjugate may hydrophobically collapse and aggregate, forming a separate phase. When there is a plurality of conjugates, the plurality of conjugates can phase transition above their Tₜ's to form an aggregate that includes the plurality of conjugates.

The phase of the conjugate may be described as, for example, soluble or an aggregate. The aggregate may be a variety of forms. The form and size of the aggregate may depend on the temperature, the composition of the copolymer, or a combination thereof. The aggregate may be, for example, nanoscale aggregates, micron-sized aggregates, or macroscale aggregates. In some embodiments, at a temperature above the Tₜ the aggregate has a diameter or length of about 1 μm to about 1 cm. In some embodiments, the aggregate is a coacervate.

The conjugate may have a varying Tₜ depending on its application. The conjugate may have a Tₜ of about 0° C. to about 100° C., such as about 10° C. to about 50° C., or about 20° C. to about 42° C. In some embodiments, the conjugate has a Tₜ of room temperature (about 25° C.) to body temperature (about 37° C.). In some embodiments, the conjugate has a Tₜ of about 28° C. to about 32° C. In some embodiments, the conjugate has its Tₜ below body temperature at the concentration at which the conjugate is administered to a subject. The Tₜ of the conjugate can depend on the molecular weight of the copolymer of POEGMA, monomer composition of the copolymer of POEGMA, the conjugate's concentration, or a combination thereof. Accordingly, the Tₜ can be adjusted by varying the aforementioned parameters and properties. In addition, the Tₜ of the conjugate can be measured by optical density via a UV-vis spectrophotometer as described in the Examples.

The conjugate may undergo phase transition at varying concentrations. For example, the conjugate may phase transition at a concentration of about 1 μM to about 1 M, such as about 10 μM to about 500 μM, about 15 μM to about 250 μM, about 20 μM to about 150 μM, or about 25 μM to about 100 μM. In some embodiments, the conjugate phase transitions at a concentration that is suitable for administration to a subject. In some embodiments, the conjugate has a Tₜ of about 0° C. to about 100° C. at a concentration of about 1 μM to about 1 M, a Tₜ of about 10° C. to about 50° C. at a concentration of about 1 μM to about 1 M, a Tₜ of about 20° C. to about 42° C. at a concentration of about 1 μM to about 1 M, a Tₜ of about 25° C. to about 37° C. at a concentration of about 1 μM to about 1 M, or a Tₜ of about 28° C. to about 32° C. at a concentration of about 1 μM to about 1 M. In some embodiments, the conjugate has a Tₜ of about 0° C. to about 100° C. at a concentration of about 500 μM, a Tₜ of about 10° C. to about 50° C. at a concentration of 500 μM, a Tₜ of about 20° C. to about 42° C. at a concentration of about 500 μM, a Tₜ of about 25° C. to about 37° C. at a concentration of about 500 μM, or a Tₜ of about 28° C. to about 32° C. at a concentration of about 500 μM.

### A. Copolymers of POEGMA

The copolymer of POEGMA can instill the conjugate with advantageous stealth, immune system, and phase transition properties. The POEGMA has a poly(methacrylate) backbone and a plurality of side chains covalently attached to the backbone. The side chains are oligomers of ethylene glycol (EG). The length of each side chain is dependent on the monomers used to synthesize the copolymer of POEGMA. For example, the disclosed copolymer of POEGMA includes monomers that provide side chains that include 2 monomers of EG repeated in tandem and 3 monomers of EG repeated in tandem. Accordingly, the copolymer of POEGMA can have a plurality of side chains covalently attached to the backbone, wherein the plurality of side chains includes a first set of side chains having 2 monomers of EG repeated in tandem, and a second set of side chains having 3 monomers of EG repeated in tandem. In addition, the oligoethylene glycol side chains may include a first end and a second end. The first end may be attached to the backbone and the second end may include a capping moiety. The capping moiety may be hydroxyl or C₁-C₃ alkyl. In some embodiments, the capping moiety is a C₁-C₃ alkyl.

In some embodiments, the copolymer is derived from monomer units of

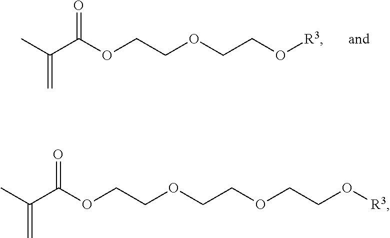

wherein R³ is hydrogen, C₁-C₃ alkyl, C₁-C₄ alkylenyl-OH, or a combination thereof.

The copolymer of POEGMA can be derived from varying amounts of the above monomers. For example, the copolymer of POEGMA can be derived from about 1 molar % to about 99 molar %, such as about 20 molar % to about 85 molar % or about 40 molar % to about 75 molar %, monomer units of

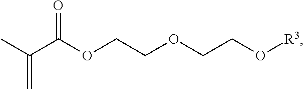

and about 1 molar % to about 99 molar %, such as about 10 molar % to about 75 molar % or about 25 molar % to about 60 molar %, monomer units of

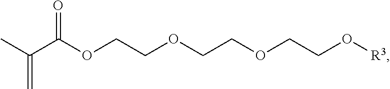

where R³ is as described above.

In some embodiments, the copolymer of POEGMA includes recurring units of formula (1):

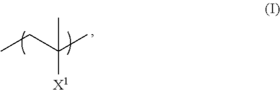

wherein X¹ is of formula (II)

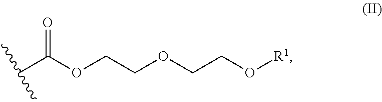

or formula (III)

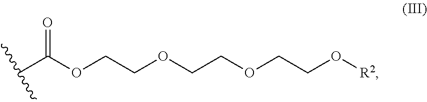

wherein R¹ and R² are each independently hydrogen, alkyl, ester, C₁-C₄ alkylenyl-NH₂, amide, carboxyl, or CI-C₄ alkylenyl-OH.

The copolymer of POEGMA can have oligoethylene glycol side chains with varying terminal end groups (e.g., hydroxy, methyl, etc.). As listed above, in some embodiments, R¹ and R² are each independently hydrogen, alkyl, ester, C₁-C₄ alkylenyl-NH₂, amide, carboxyl, or C₁-C₄ alkylenyl-OH. In some embodiment, R¹ and R² are each independently hydrogen, C₁-C₃ alkyl, carboxyl, or C₁-C₄ alkylenyl-OH. In some embodiments, R¹ and R² are each independently hydrogen, C₁-C₃alkyl, or C₁-C₄ alkylenyl-OH. In some embodiments, R¹ and R² are each independently hydrogen or methyl.

The copolymer of POEGMA can include recurring units with formula (II) and formula (III) at varying amounts. For example, the copolymer of POEGMA can include about 1 molar % to about 99 molar % of recurring units with formula (II), such as about 20 molar % to about 85 molar % or about 40 molar % to about 75 molar %. The copolymer of POEGMA can also include about 1 molar % to about 99 molar % of recurring units with formula (III), such as about 10 molar % to about 75 molar % or about 25 molar % to about 60 molar %. The amount of each type of recurring unit can be modulated by altering the amount of each corresponding monomer added to the reaction. In addition, the amount of each type of recurring unit can be measured by NMR as described in the Examples.

The copolymer of POEGMA can be any suitable type of copolymer that still is able to provide the properties of the disclosed conjugate. For example, the copolymer of POEGMA can be a random copolymer, a block copolymer, or an alternating copolymer.

The copolymer of POEGMA can have varying amounts of the recurring units of formula (I). For example, the copolymer of POEGMA can have about 100 to about 1,000 recurring units of formula (I), such as about 100 to about 600 recurring units of formula (I), about 100 to about 400 recurring units of formula (I), or about 200 to about 300 recurring units of formula (I).

The copolymer of POEGMA can have a varying molecular weight. For example, the copolymer of POEGMA can have a weight average molecular weight of about 2 kDa to about 500 kDa, such as about 5 kDa to about 300 kDa, about 10 kDa to about 200 kDa, about 15 kDa to about 100 kDa, or about 20 kDa to about 60 kDa. Molecular weight of the copolymer can be measured by techniques used within the art, such as size-exclusion chromatography (SEC), SEC combined with multi-angle light scattering, gel permeation chromatography, and the like.

The copolymer of POEGMA can also have phase transition properties when not conjugated to the biologically active agent. Accordingly, the copolymer of POEGMA may have a Tₜ when not conjugated to the biologically active agent.

Further discussion on POEGMA and it application can be found in U.S. Pat. Nos. 8,497,356 and 10,364,451, both of which are incorporated herein by reference in their entirety.

### B. Biologically Active Agents

The conjugate includes a biologically active agent. A large variety of different biologically active agents may be used with the copolymers of the disclosure Examples include, but are not limited to, a monoclonal antibody, blood factor, betatrophin, exendin, enzyme, asparaginase, glutamase, arginase, arginine deaminase, adenosine deaminase (ADA), ADA-2, ribonuclease, cytosine deaminase, trypsin, chymotrypsin, papain, growth factor, epidermal growth factor (EGF), insulin, insulin-like growth factor (IGF), transforming growth factor (TGF), nerve growth factor (NGF), platelet-derived growth factor (PDGF), bone morphogenic protein (BMP), fibroblast growth factor (FGF), somatostatin, somatotropin, somatropin, somatrem, calcitonin, parathyroid hormone, colony stimulating factors (CSF), clotting factors, tumor necrosis factors (TNF), gastrointestinal peptides, vasoactive intestinal peptide (VIP), cholecystokinin (CCK), gastrin, secretin, erythropoietins, growth hormone, GRF, vasopressins, octreotide, pancreatic enzymes, superoxide dismutase, thyrotropin releasing hormone (TRH), thyroid stimulating hormone, luteinizing hormone, luteinizing hormone-releasing hormone (LHRH), growth hormone releasing hormone (GHRH), tissue plasminogen activators, interleukins, interleukin-1, interleukin-15, interleukin-2, interleukin-10, colony stimulating factor, granulocyte macrophage colony-stimulating factor (GM-CSF), interleukin-1 receptor antagonist (IL-IRA), glucagon-like peptide-1 (GLP-1), exenatide, GLP-1 R multi-agonist, GLP-1 R antagonist, GLP-2, TNF-related apoptosis-inducing ligand (TRAIL), leptin, ghrelin, granulocyte monocyte colony stimulating factor (GM-CSF), interferons, interferon-α, interferon-gamma, human growth hormone (hGH) and antagonist, macrophage activator, chorionic gonadotropin, heparin, atrial natriuretic peptide, hemoglobin, relaxin, cyclosporine, oxytocin, vaccines, monoclonal antibodies, single chain antibodies, ankyrin repeat proteins, affibodies, activin receptor 2A extracellular domain, alpha-2 macroglobulin, alpha-melanocyte, apelin, bradykinin B2 receptor antagonist, cytotoxic T-lymphocyte-associated protein (CTLA-4), elafin, Factor IX, Factor VIIa, Factor VIII, hepcidin, infestin-4, kallikrein inhibitor, L4F peptide, lacritin, parathyroid hormone (PTH), peptide YY (PYY), thioredoxin, thymosin B4, urate oxidase, urodilatin, aptamers, silencing RNA, microRNA, long non-coding RNA, ribozymes, analogs and derivatives thereof, and combinations thereof.

In some embodiments, the biologically active agent includes a nucleotide, a polynucleotide, a protein, a peptide, a polypeptide, a carbohydrate, a lipid, a small molecule drug, or a combination thereof. In some embodiments, the biologically active agent includes a nucleotide, a polynucleotide, a protein, a peptide, or a polypeptide. In some embodiments, the biologically active agent includes a protein, a peptide, or a polypeptide. In some embodiments, the biologically active agent includes a polypeptide.

### C. Conjugation

The copolymer of POEGMA can be conjugated to the biologically active agent by conjugation strategies known within the art. For example, the biologically active agent and the copolymer of POEGMA may each individually have functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, thiols for formation of disulfides, an aldehyde and amine for enamine formation, an azide for formation of an amide via a Staudinger ligation. Functional groups suitable for conjugation also include bioorthogonal functional groups. Bioorthogonal functional groups can selectively react with a complementary bioorthogonal functional group. Bioorthogonal functional groups include, but are not limited to, an azide and alkyne for formation of a triazole via Click-chemistry reactions, trans-cyclooctene (TCO) and tetrazine (Tz) (e.g., 1,2,4,5-tetrazine), and others. In some embodiments, the biologically active agent and the copolymer of POEGMA each individually include bioorthogonal functional groups. In some embodiments, the biologically active agent is functionalized with dibenzocyclooctyne, the copolymer of POEGMA is functionalized with an azide, or both. In addition, the biologically active agent can be conjugated to the copolymer of POEGMA such that it retains its biological action.

In some embodiments, the biologically active agent is conjugated to the copolymer of POEGMA via a triazole. In some embodiments, the biologically active agent is conjugated to the backbone of the POEGMA. In some embodiments, the biologically active agent is conjugated to the backbone of the copolymer of POEGMA via a triazole.

### 3. Uses of the Conjugates

### A. Compositions

Also disclosed are uses of the conjugates. As mentioned above, the conjugates have temperature dependent phase transition behavior. Phase transition behavior may be used to form drug depots within a tissue of a subject for controlled (slow) release of the conjugate. For example, a plurality of conjugates can self-assemble into an aggregate above the Tₜ of conjugate. Accordingly, also disclosed herein are compositions that include a plurality of conjugates, wherein the plurality of conjugates self-assemble into an aggregate above the Tₜ of the conjugate. The plurality of conjugates can include conjugates having the same Tₜ or can include conjugates having a range of Tₜ's. In some embodiments, the aggregate including a plurality of self-assembled conjugates is referred to as a drug depot.

i. Administration

The disclosed compositions may be incorporated into pharmaceutical compositions suitable for administration to a subject (such as a patient, which may be a human or non-human) well known to those skilled in the pharmaceutical art. The pharmaceutical composition may be prepared for administration to a subject. Such pharmaceutical compositions can be administered in dosages and by techniques well known to those skilled in the medical arts taking into consideration such factors as the age, sex, weight, and condition of the particular subject, and the route of administration.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols, such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the composition is administered and the form of the composition can dictate the type of carrier to be used.

The composition can be administered prophylactically or therapeutically In prophylactic administration, the composition can be administered in an amount sufficient to induce a response. In therapeutic applications, the composition can be administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The compositions can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), which are all incorporated by reference herein in their entirety. One skilled in the art would know that the choice of a pharmaceutically acceptable carrier, including a physiologically acceptable compound, depends, for example, on the route of administration.

The composition may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions can be administered using a regimen which maintains plasma levels above the MEC for 10-90% of the time, such as between 30-90% or between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### B. Methods of Treating Diseases

The present disclosure also provides methods of treating a disease. The methods include administering to a subject (in need thereof) an effective amount of the composition as detailed herein, e.g., including a plurality of conjugates, wherein the plurality of conjugates self-assemble into an aggregate above the Tₜ of the conjugate.

The disclosed methods can take advantage of the conjugate's phase transition behavior. For example, the compositions can be administered at a temperature below the Tₜ of the conjugate to an area of the subject that has a temperature above the conjugate's Tₜ. Practically speaking, this can allow the compositions to be administered in liquid form through, e.g., a syringe to a subject, and then following injection, the composition can phase transition to an aggregate (e.g., depot) at the site of administration. The aggregate/depot can restrict the release of conjugate and/or agent and thus sustain its release over a longer period of time. For example, the aggregate can allow the conjugate to be released over an extended period of time, such as about 12 hours to about 3 months following administration. In some embodiments, the composition releases the conjugate following administration for greater than 3 days, greater than 7 days, greater than 2 weeks, greater than 1 month, or greater than 3 months. In some embodiments, the composition releases the conjugate following administration for less than 3 months, less than 2 months, less than 1 month, less than 2 weeks, or less than 10 days. In some embodiments, the composition releases the conjugate following administration for greater than 7 days.

Similar to the conjugates, the compositions can also have advantageous immune properties. For example, following administration, the composition can have a reduced immune response relative to a polyethylene glycol (PEG)-biologically active agent conjugate; may not induce an anti-POEGMA antibody response; may not react with pre-existing anti-PEG antibodies in the subject; or a combination thereof. In addition, the composition may also not induce a histopathological change in the subject. For example, the composition may not induce any histopathological changes (e.g., vacuolization) in a subject's organs, such as the kidney or pancreas. In some embodiments, the composition does not induce vacuolization in the subject. In some embodiments, the composition does not induce vacuolization in the kidneys, pancreas, or both of the subject. Immune response and histopathological effect can be assessed as described in the Examples.

The compositions can be administered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the composition is administered subcutaneously, intradermally, intramuscularly, or intraperitoneally.

Many types of diseases can be treated by the disclosed conjugates and compositions thereof. Examples include, but are not limited to, cancer, metabolic diseases, autoimmune diseases, cardiovascular diseases, and orthopedic disorders. In some embodiments, the disease is a cancer or a metabolic disease.

Metabolic diseases may occur when abnormal chemical reactions in the body alter the normal metabolic process. Metabolic diseases may include, for example, obesity, type 2 diabetes mellitus, pancreatitis, dyslipidemia, nonalcoholic fatty liver disease (NAFLD), nonalcoholic steatohepatitis (NASH), insulin resistance, hyperinsulinemia, glucose intolerance, hyperglycemia, and glucose metabolic disorders.

In some embodiments, the disease is a metabolic disorder and the administered composition can demonstrate advantageous results. For example, the administration of the compositions can result in the subject having at least one of decreased blood glucose, decreased body fat, increased insulin production, decreased hemoglobin A1c values, decreased circulating fatty acids, decreased liver fat content, decreased liver inflammation, and/or decreased liver fibrosis compared to the subject not receiving the administration of the composition. In some embodiments, the subject has decreased blood glucose for at least 6 days, such as at least 7 days, at least 10 days, at least 14 days, or at least 21 days, after a single administration of the composition compared to the subject not receiving the administration of the composition.

Autoimmune diseases arise from an abnormal immune response of the body against substances and tissues normally present in the body. Autoimmune diseases may include, but are not limited to, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, polyarteritis nodosa, myocarditis, psoriasis, Celiac disease, Crohn's disease, ulcerative colitis, and fibromyalgia.

Cardiovascular disease is a class of diseases that involve the heart or blood vessels. Cardiovascular diseases may include, for example, coronary artery diseases (CAD) such as angina and myocardial infarction (heart attack), stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

Orthopedic disorders or musculoskeletal disorders are injuries or pain in the body's joints, ligaments, muscles, nerves, tendons, and structures that support limbs, neck, and back. Orthopedic disorders may include degenerative diseases and inflammatory conditions that cause pain and impair normal activities. Orthopedic disorders may include, for example, carpal tunnel syndrome, epicondylitis, and tendinitis.

Cancers may include, but are not limited to, breast cancer, colorectal cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

The description of the conjugates, compositions, biologically active agent, and copolymer of POEGMA can also be applied to the uses and methods disclosed herein.

The present invention has multiple aspects, illustrated by the following non-limiting examples.

### EXAMPLES

### Example 1

### Materials & Methods

Synthesis and purification of POEGMA. All materials were purchased from Millipore Sigma. Triethylene glycol methyl ether methacrylate (EG3) and diethylene glycol methyl ether methacrylate (EG2) were passed through basic alumina columns to remove inhibitors. Other materials were used as received. A catalytic complex was prepared by mixing tris(2-pyridylmethyl) amine (TPMA) and copper (II) bromide (CuBr2) in ultrapure water at a final concentration of 0.8M and 0.1M, respectively. In a typical copolymerization, a Schlenk flask contained EG3 (3.5 mmol; 701.57 μl), EG2 (6.5 mmol; 1199.44 μl), azide functional polymerization initiator (0.2M in methanol; 125 μl), the catalytic complex (62.5 μl), methanol (5.875 ml) and 100 mM NaCl (11.946 ml). The polymerization flask was sealed and cooled to 0° C. in an ice bath. A separate Schlenk flask contained 64 mM ascorbic acid in ultrapure water. Both flasks were purged with argon for 45 minutes on ice to remove oxygen. After deoxygenation, the ascorbic acid solution was continuously injected into the polymerization flask at a rate of 1 μl min⁻¹ using a syringe pump under an inert atmosphere. The resulting solution was kept under vacuum to remove methanol and freeze-dried overnight. The resulting POEGMA was solubilized in acetonitrile and passed through a neutral alumina column to remove the catalytic complex. POEGMA was purified from unreacted monomer by precipitation in cold diethyl ether, followed by overnight evaporation of excess diethyl ether under vacuum.

Physical characterization of POEGMA. Mₙ, M_{w}, and Ð of POEGMA were assessed by GPC-MALS. POEGMA was solubilized in tetrahydrofuran (THF) at 2 mg ml-1, followed by filtration through a 0.22 μm Teflon syringe filter. 50 μl of the solution was separated on an Agilent PLgel mixed-C column (105 Å, 7.5 mm internal diameter×300 mm length, and 5 μm particle size) using an Agilent 1100 analytical high-pressure liquid chromatography (HPLC). The HPLC was equipped with a UV detector operating at 254 nm (Agilent), a Dawn EOS MALS detector (Wyatt Technology), and an Optilab DSP refractometer (Wyatt Technology). The mobile phase included 100 ppm butylated hydroxytoluene (BHT) in THF as a stabilizer. The flow rate was 1 ml min⁻¹. The MALS detector was annually calibrated in toluene and normalized with 30 kDa polystyrene (Wyatt Technology) before each analysis. Refractive index increment (dn/dc) of POEGMA was calculated using a built-in method on ASTRA software (v. 6.0, Wyatt Technology) based on injections of known concentrations and mass, followed by data analysis for Mₙ, M_{w}, and Ð.

Structural characterization of POEGMA. POEGMA structure was characterized by H-NMR spectroscopy using a 400 MHz Varian INOVA spectrometer and ACD/NMR software (ACD Labs). The monomer composition was defined as the percentage of EG2 (or EG3) content in the copolymer. The monomer composition was calculated from the integral value that corresponds to the average number of hydrogens (H) present in the OEG side-chain (b; 3.4-4.4 ppm; 6H for EG2_{100%} homopolymer; 10H for EG3_{100%} homopolymer) except chain end-group (c; 3.5-3.3 ppm; 3H) and methylene protons (a; 4.0-4.4 ppm; 2H). DP were calculated by subtracting the polymerization initiator's M_{w} from POEGMA's M_{w} and dividing the resulting mass by the average M_{w} of a monomeric unit.

Hydrodynamic size characterization. Rₕ of POEGMA and exendin-POEGMA conjugates was characterized by DLS in PBS at 1 mg ml⁻¹ using a temperature-controlled DynaPro Plate Reader (Wyatt Technology). Samples were filtered through a 100 nm syringe filter (Whatman). Ten repeat measurements of 10-second acquisitions were made at 15° C. Data were analyzed for Raleigh spheres by applying a regularization fit using Dynamics 6.12.0.3 software (Wyatt Technology). The laser wavelength and scattering angle of the instrument were 831.95 nm and 90°, respectively.

Phase behavior characterization. The optical density of POEGMA and exendin-POEGMA conjugates was monitored at 600 nm in PBS at pH 7.4 as the temperature gradually increased at a rate of 1° C. min⁻¹ using a temperature-controlled UV-vis spectrophotometer (Cary 300 Bio, Varian Instruments). A sharp increase in optical density as temperature increased indicated the phase transition. The Tₜ was defined as the inflection point of temperature versus the optical density curve and calculated as the maximum of the first derivative using GraphPad Prism 8.0 software. Reversibility of the phase behavior was shown by monitoring the optical density as the temperature gradually decreased.

Protein expression and purification. Exendin was expressed in E. coli as an ELP fusion protein with a sortase-A recognition site (LPETG) and polyhistidine tag, yielding exendin-LPETG-His₆-ELP (ESE). The ELP tag enables rapid non-chromatographic purification of ESE, while LPETG peptide acts as the sortase ligation site. Both ESE and His₆-Sortase A were expressed and purified to >95% purity. Purity was assessed by sodium dodecyl sulfate-polyacrylamide gel electrophoresis (SDS-PAGE) using 4-20% precast Tris-HCl gels (Bio-Rad), followed by staining in Simply Blue Safe Stain (Thermo Scientific) and gel densitometry analysis using Image Lab software (Bio-Rad). ESE concentration was measured using Bicinchoninic Acid (BCA) assay (Pierce) according to the manufacturer's instructions. His₆-Sortase concentration A was measured by UV-visible spectroscopy using an ND-1000 Nanodrop spectrophotometer (Thermo Scientific).

Synthesis, purification, and characterization of exendin-DBCO. A bio-orthogonal DBCO group was installed on the C-terminus of exendin via sortase A-mediated native peptide ligation, yielding exendin-DBCO. Briefly, ESE (100 μM) and His₆-Sortase A (50 μM) were reacted in the presence of triglycine-DBCO (Gly₃-DBCO) (5 mM; Click Chemistry Tools) in ligation buffer (50 mM Tris, 150 mM NaCl, and 10 mM CaCl₂; pH 7.5) at room temperature for 16 hours. The resulting solution was purified by reverse immobilized metal affinity chromatography using an AKTA Purifier (GE Healthcare) equipped with a photodiode array operating at 220 and 280 nm and HisTrap HP (GE Healthcare) columns. Exendin-DBCO was collected in the flow-through as it was the only species that did not carry a polyhistidine tag, thereby showing no binding to the resin. Exendin-DBCO was concentrated by ultrafiltration using Centricon 70 (Millipore Sigma) filters with a 3,000 Da molecular weight cut-off (MWCO), followed by dialysis into cold water and lyophilization. Stoichiometric (1:1) attachment of DBCO to exendin was confirmed by Matrix-Assisted Laser Desorption/Ionization-Time-of-Flight mass spectroscopy (MALDI-TOF-MS).

Synthesis, purification, and characterization of an azide-functional amide-based polymerization initiator. 2-Bromoisobutanoic acid N-hydroxy succinimide ester (4.1 mmol; 1.14 g) was purged with argon and dissolved in 5 ml anhydrous dichloromethane (DCM) (Flask 1). In a separate Schlenk flask (Flask 2), 3-azido-1-propanamine (4.9 mmol; 0.52 g) was purged with argon and dissolved in 11.09 ml anhydrous DCM, followed by cooling to 0° C. in an ice bath. The solution in Flask 1 was then dropwise added to Flask 2 under an inert atmosphere. The resulting solution was kept on ice for 30 minutes and left stirring at 30° C. for 12 hours. The solution was diluted in DCM and passed through a polyvinylidene fluoride (PVDF) membrane to remove the solid phase. The resulting clear solution was washed with 0.5N HCl, saturated Na₂HCO₃, and 1M NaCl, respectively, and the organic phase was collected. The organic phase dried over anhydrous MgSO₄, followed by filtration through a PVDF membrane and DCM evaporation under vacuum, yielding the polymerization initiator. The polymerization initiator was characterized using high-resolution mass and nuclear magnetic resonance (NMR) spectrometry.

Synthesis and purification of exendin conjugates. Exendin-DBCO was conjugated to azide functional POEGMA or PEG via strain-promoted alkyne-azide click reaction. Exendin-DBCO and azide functional POEGMA or PEG were dissolved in PBS at a 1.05:1 ratio and reacted overnight at 4° C. Depot-forming exendin-POEGMA conjugates were purified by triggering phase transition with the addition of ammonium sulfate to a final concentration of 0.1 M. The phase-transitioned conjugate was recovered by centrifugation at 21,000 g for 15 minutes at room temperature, the supernatant was removed, and the conjugate was dissolved in PBS at 4° C. The last two steps were repeated two more times to obtain conjugates with purity greater than 99%, verified by SDS-PAGE and HPLC. In the final step, the conjugate was dissolved in ultra-pure water and lyophilized. Soluble exendin-POEGMA and exendin-PEG conjugates were purified via a single round of size exclusion chromatography (SEC) using an AKTA purifier equipped with a photodiode array detector operating at 220 and 280 nm and a HiLoad 16/600 Superdex 75 pg column (GE Healthcare) at 4° C. using PBS as the mobile phase. The purified conjugates were concentrated by ultrafiltration using Amicon filters (Millipore Sigma) with 3,000 Da MWCO, followed by dialysis into the water at 4° C. overnight and lyophilization.

Physical characterization of exendin-POEGMA conjugates. The conjugates were characterized in terms of their Mₙ, M_{w}, and Ð by SEC-MALS using an Agilent 1260 analytical HPLC equipped with a UV detector operating at 280 nm (Agilent), a DAWN HELEOS II MALS detector (Wyatt Technology), and an Optilab T-rEX refractive index detector (Wyatt Technology). DAWN HELEOS II MALS detector was annually calibrated in toluene and normalized with 2 mg ml⁻¹ bovine serum albumin (Pierce) before each analysis. An exendin-POEGMA conjugate was dissolved in 10 mM phosphate buffer at pH 7.4, followed by filtration through a 100 nm syringe filter (Whatman). 50 μl of the conjugate was separated on a Shodex KW-803 column (8 mm internal diameter×300 mm length, and 5 μm particle size). The mobile phase was 30% (v/v) methanol in 10 mM phosphate buffer at pH 7.4. The flow rate was 0.5 ml min⁻¹. Data were analyzed for Mₙ, M_{w}, and Ð using a build-in protein conjugate method on ASTRA software (v. 7.0, Wyatt Technology).

In vitro activity of exendin variants. Exendin variants' activity was tested in a cell-based assay in terms of GLP1R activation, which increases intracellular cyclic adenosine monophosphate (cAMP) levels. Intracellular cAMP concentrations were quantified by treating Human Embryonic Kidney 293 cells, which recombinantly express GLP1R and luciferase fused CAMP (HEK293/CRE-Luc/GLP1R), with exendin variants.

HEK293/CRE-Luc/GLP1R cells were cultured in high glucose Dulbecco's Minimal Essential Medium (DMEM) (Gibco), supplemented with 10% fetal bovine serum (Hyclone), 400 μg ml⁻¹ G418 (Thermo Fisher), and 200 μg ml⁻¹ Hygromycin B (Invitrogen). Cells were subcultured at least once before the assay at approximately 80% confluency. One day before performing the assay, cells were seeded without antibiotics in phenol red-free DMEM (Gibco) on 96-well plates at 25,000 cells per well in 90 μl media followed by incubation at 37° C. under 5% CO₂ atmosphere overnight. Exendin conjugates (20 μM in PBS) were incubated with dipeptidyl peptidase IV (DPP-IV, Prospec Bio) to expose an active N-terminus for 16 h at room temperature. DPP-IV amount was 2.5 mass % of exendin present in the conjugates. On the day of the assay, exendin (Santa Cruz Biotechnology) was dissolved to a final concentration of 20 μM in PBS. Logarithmic serial dilutions were made for exendin variants in PBS using unmodified exendin as a positive control. 10 μl of each dilution was transferred to wells (n=6), yielding a concentration range of 10⁻¹³-10⁻⁶ M. The plates were then incubated at 37° C. for 5 hours, followed by equilibration with room temperature for 1 hour. 100 μl Bright-Glo™ reagent (Promega) was added to the wells and incubated for 2 minutes, followed by measuring luminescence using a Victor plate reader (Perkin Elmer). Data were analyzed for net luminescence by subtracting PBS-treated wells' mean luminescence (negative control) from exendin variants. The effective half-maximal dose (EC₅₀) of each exendin variant was determined by fitting the dose-response curve to a four-parameter logistic, nonlinear regression model using GraphPad Prism 8 software.

In vivo studies. In vivo studies were conducted under protocols approved by Duke Institutional Animal Care and Use Committee (IACUC) by employing six-week-old male C₅₇BL/6J (Jackson Laboratories; stock no. 000664) or B6.BKS(D)-LepRdb/J mice (db/db; Jackson Laboratories; stock no: 000697). C₅₇BL/6J mice were kept on a 60 kilocalorie (kcal) % fat diet (Research Diets Inc.; #D12492i) for at least five weeks before and during the experiments unless otherwise noted, yielding DIO mice. Six-week-old male db/db mice were fed a standard rodent diet (LabDiet 5001) and acclimatized to facilities for one week before the experiments. In immunogenicity experiments, mice treated with OVA variants were kept on a standard rodent diet. Mice were group-housed under controlled photoperiod with 12 h light and 12 h dark cycles and acclimated to the facility for a week before the start of experiments. Mice had ad libitum access to water and food unless otherwise noted.

All samples were endotoxin purified using endotoxin removal columns (Pierce) and sterilized using a 0.22 μm Acrodisc filter with a Mustang E membrane (Pall Corporation). The final endotoxin amount was tested below 5 EU per kg mouse body weight using the Endosafe nexgen-PTS instrument and cartridges (Charles River). For the samples used in the immunogenicity and the long-term efficacy experiments, a more stringent endotoxin limit of a maximum of 0.2 EU per kg mouse body weight was used.

Fed blood glucose measurements. In the short-term efficacy experiments, the fed blood glucose was measured after a single s.c. injection of the treatments. On the day of injection, the tail was sterilized with alcohol pads (BD). The first drop of blood collected from a tiny incision on the tail vein was wiped off. The second drop of blood was used to measure fed blood glucose using a hand-held glucometer (Alpha Track, Abbott). The treatments were solubilized in PBS and kept on ice before injection to prevent phase transition, followed by s.c. administration into mice. Bydureon was prepared for injection according to the manufacturer's instructions. Fed blood glucose levels were measured 24 h and immediately before injection, at 1-, 4-, and 8-h post-injection, and every 24 h after that until no significant effect of treatments on fed blood glucose was observed. Body weight was tracked daily. In the long-term efficacy experiment, fed blood glucose and body weight were tracked every three days.

Intraperitoneal glucose tolerance test (IPGTT). The glycemic regulation ability of treatments was assessed by performing three IPGTTs after a single s.c. injection. Db/db mice were used during the first week of the long-term pharmacodynamics study. On day 0, the treatments were administrated into the mice at an equivalent dose (1000 nmol per kg bodyweight) and concentration (500 μM) using the equivalent injection volume of PBS as a negative control. Bydureon was prepared for injection according to the manufacturer's instructions. On the day of the IPGTT (Day 1, 3, and 5), mice were fasted six hours before the glucose challenge by an intraperitoneal (i.p) injection of 1.5 g kg⁻¹ of glucose (Sigma) followed by blood glucose monitoring at 5-, 15-, 30-, 60-, 90-, 120-, 180- and 240 min.

HbA1c % measurement. In long-term pharmacodynamics experiments, db/db mice (n=5) were repeatedly injected with the treatments at an equivalent dose (1000 nmol per kg body weight in PBS) and concentration (500 μM) every seven days over 56 days using an equivalent injection volume of PBS as a negative control. Bydureon was prepared for injection according to the manufacturer's instructions. HbA1c % was measured on Day 0 before injection, Day 28, and Day 56 using DCA Vantage Analyzer and DCA HbA1c reagent kit (Siemens).

Pharmacokinetics. Exendin variants were labeled with a fluorophore to track their pharmacokinetics. Briefly, Alexa Fluor 488 NHS ester (Pierce) was reacted with exendin variants (5 mg ml⁻¹) at a 5:1 molar ratio in PBS for one hour at room temperature. Unreacted excess fluorophore was removed by dialysis into the water at 4° C. using membranes with a 3,000 Da MWCO (Pierce), verified by HPLC. The labeling efficiency was calculated from UV-vis spectroscopy using an ND-1000 Nanodrop spectrophotometer (Thermo Scientific).

The fluorophore-labeled treatments were administered into DIO C₅₇BL/6J mice via a single s.c. injection at 1000 nmol kg⁻¹ (45 nmol kg⁻¹ fluorophore). Ten μl of blood was collected from a tiny incision on the tail vein into tubes containing 90 μl of 1,000 U ml⁻¹ heparin (Sigma) at 5-min, 1-, 2-, 4-, 8-, 24-, 48-, 72-, 96-, 120-, 144- and 168-h. Blood samples were centrifugated at 1600 g at 4° C. for 15 minutes for plasma. Fluorophore concentration in plasma samples was detected by a Victor plate reader (Perkin Elmer) at 485 nm (excitation) and 535 nm (emission). PK parameters were derived by plotting the drug's plasma concentration as a function of time and fitting it to a non-compartmental PK model for the absorption and elimination phases using GraphPad Prism software. The absorption phase described the time between injection at 0-h and tₘₐₓ, where the drug concentration was maximum. The elimination phase described the time after tₘₐₓ. tₘₐₓ was calculated from equation

$${t}_{max} = ( \frac{2.303}{{k}_{a} - {k}_{e}} ) * log \frac{{k}_{a}}{{k}_{e}} ,$$

where kₐ and kₑ were the apparent absorption and elimination rate constants. The rate constants were determined from the linear regression slope of the log (drug concentration) versus time graph using equation k=−2.303*slope. Half-lives of treatments in each phase were determined from the equation

$${t}_{1 / 2} = \frac{0.693}{k} .$$

The maximum drug concentration (Cₘₐₓ) was calculated at tₘₐₓ. Minimal effective conjugate concentration was calculated by triangulating the concentration values based on the duration of blood glucose control determined in short-term efficacy experiments in DIO mice.

Dose optimization of Ex-POEGMAₒₚₜ. In the dose optimization study, varied doses of Ex-POEGMAₒₚₜ were administrated into 11-weeks-old diet-induced obese C₅₇BL6/J mice (n=5) that were kept on a 60 kilocalorie % fat diet for five weeks before the study. Injection concentration (500 μM) and volume (120 μl) were kept constant across groups to prevent differences in Tₜ by bringing the volume of lower dose injections to that of the highest dose using free POEGMA (500 μM). Fed blood glucose and body weight were tracked at 1-, 4-, 8-, 24-, 48-, 72-, 96-, 120-, 144- and 168 h post-injection. Increased injection doses resulted in more extended blood glucose control. Because an injection dose of 1500 nmol kg⁻¹ did not provide an additional benefit, 1000 nmol kg⁻¹ injection dose.

ADAs' effect on the PK was investigated by weekly administering the fluorophore-labeled treatments into the DIO C₅₇BL/6J mice five times. Blood samples were collected after the first and last injection, and PK parameters were determined as described above.

Immunogenicity. Immunogenicity of POEGMA was tested and compared to that of PEG in three sets of immunogenicity experiments. In the first set, sterile and endotoxin-free exendin, Ex-PEG_{Mw}, and Ex-POEGMAₒₚₜ were s.c. administrated into 7-week-old C₅₇BL/6J mice (n=10) at an equivalent dose (1000 nmol kg⁻¹) and concentration (500 μM). In the second set of experiments, mice were injected with OVA (Invivogen), OVA-PEG_{10K}, and OVA-POEGMA_{10K} at 9.6 nmol kg⁻¹. In the final set, OVA, OVA-PEG_{10K}, and OVA-POEGMA_{10K} were emulsified in an equal volume of sterile and endotoxin-free adjuvant (Invivogen) and then administrated into 7-week-old C₅₇BL/6J mice (n=10) at 9.6 nmol kg⁻¹. Complete Freund's adjuvant (CFA) was used for the first injection, while incomplete Freund's adjuvant (IFA) was the adjuvant of choice for the rest of the injections. In all experimental sets, an equal injection volume of PBS (or PBS emulsified in CFA or IFA) was used as a negative control. All drugs were administrated into mice every 17 days three times (Day 0, 17, and 34). Blood samples were collected seven days before the first injection (Day −7) and ten days after each injection (Day 10, 27, and 44), followed by plasma isolation via centrifugation at 4° C. at 1600 g for 15 minutes and storage at −80° C. until analysis.

Analysis of anti-drug antibodies (ADA), ADAs were analyzed using a Luminex multiplex immunoassay (LMI) according to the manufacturer's instructions with minor modifications. The plasma samples of mice treated with exendin variants were diluted 200-fold in 0.2% (w/v) I-Block protein-based blocking reagent (Thermo Scientific) in PBS (Hyclone), defined as the assay buffer. The plasma samples of mice treated with OVA variants (n=10) were diluted 500- and 10000-fold in the assay buffer for IgM and IgG analysis, respectively.

In a typical LMI assay, 50 μl of the diluted plasma samples (n=3) were transferred to a black, round-bottom 96-well-plate (Corning). Next, spectrally distinct exendin-, exendin-PEG-, exendin-POEGMA-, OVA-, OVA-PEG-, and OVA-POEGMA-coupled magnetic bead sets were mixed at a concentration of 2500 beads per 50 μl per set. 50 μl of the resulting solution was distributed to the wells of a 96-well plate and incubated for 1 hour on an orbital shaker to capture ADAs present in the plasma samples. After incubation, 96-well-plate was placed on a magnetic ring stand (Invitrogen) and incubated for 60 seconds for separation to occur. The supernatant was discarded, and wells were washed with 100 μl of the assay buffer twice. The resulting magnetic bead-ADA complexes were incubated with 100 μl of 5 μg ml⁻¹ R-Phycoerythrin-conjugated goat anti-mouse IgG (Jackson Immunoresearch; #115-115-164) for 30 minutes to detect IgG subtypes of ADAs. 100 μl of 5 μg ml⁻¹ biotin-conjugated goat anti-mouse IgM (Jackson Immunoresearch; #115-065-075) was used to detect IgM subtypes of ADAs, followed by washes, incubation with 100 μl of 7.5 μg ml⁻¹ streptavidin-R-phycoerythrin (SAPE; Invitrogen) for 30 minutes and a final round of washes. Mouse IgG- or IgM-coupled magnetic beads were used as positive controls. They were incubated with 100 μl of 0.25 μg ml⁻¹ R-Phycoerythrin-conjugated goat anti-mouse IgG or 0.114 μg ml⁻¹ biotin-conjugated goat anti-mouse IgM, followed by 0.171 μg ml⁻¹ SAPE. The negative control was the drug-coupled bead mixture in the assay buffer. Finally, magnetic beads were suspended in 100 μl of assay buffer, followed by measuring mean fluorescence signal intensity (MFI) using MAGPIX (Luminex) instrument. The average MFI value of each plasma sample and treatment was computed with no exception for outliers. Mouse plasma samples were considered ADA-positive if the average MFI for any given bead type was above the background. MFI of each plasma sample for any given bead type was normalized to the positive control.

Histology. The db/db mice used in the long-term efficacy experiment were sacrificed, and the organs were transferred into 10% neutral-buffered formalin (NBF). After fixation in 10% NBF, the tissues were paraffin embedded and sliced for light microscopy, followed by H&E staining.

Statistical Analyses. In all the animal studies, animals were randomized, and group sizes were estimated using G*Power software, and adequate power was ensured to detect differences. Data were presented as mean±standard error of the mean (SEM) unless otherwise noted differently. Blood glucose data were plotted as a function of time using raw values and normalized values. Normalization reflects the percent change in blood glucose with treatment. It was calculated by dividing raw the fed blood glucose measurement at any given time point by an average value of fed blood glucose levels measured 24 hours (t=−24 h) and immediately before injection (t=0h). Blood glucose and body weight data were analyzed using an unpaired parametric two-tailed t-test or a one-way analysis of variance (ANOVA) followed by Sidak's multiple comparison test. The area under the curve (AUC) for glucose exposure was quantified for each subject using the trapezoid rule and analyzed using two-way ANOVA, followed by Tukey's multiple comparison test. HbA1c % and immunogenicity data were analyzed using two-way ANOVA, followed by Tukey's multiple comparison test. A test was considered significant if the P-value is <0.05 (*P<0.05; **P<0.01; ***P<0.001; ****P<0.0001; ns: P>0.05). GraphPad Prism 8.0 was used for all statistical analyses.

### Example 2

### Design, Optimization, and Validation of Immunoassay & Neutralizing Antibody Assay

### Design, Optimization, and Validation of a Luminex Multiplexed Immunoassay

Assay Design. An assay was designed to assess the titer, specificity (anti-protein/peptide, anti-PEG, or anti-POEGMA), and subtype (immunoglobulin M (IgM) or IgG) of AD As using a Luminex multiplexed immunoassay (LMI) platform. The LMI platform uses drug-conjugated, fluorescently barcoded magnetic beads to capture ADAs. We conjugated exendin, exendin-PEG, and exendin-POEGMA and their ovalbumin (OVA) counterparts—OVA, OVA-PEG, OVA-POEGMA—to different sets of fluorescently barcoded magnetic beads. This bead design allowed us to determine the specificity of the ADAs, such that if exendin-PEG-treated mice plasma results in a positive signal in exendin-PEG- and OVA-PEG-conjugated bead sets but not in exendin-conjugated bead set, that would give a clear indication that ADAs were PEG-specific.

Drug synthesis purification, and characterization for bead coupling. Exendin and OVA conjugates of PEG and POEGMA were synthesized for bead coupling using polymers with an M_{w} of ˜10 kDa, yielding exendin-PEG_{10K}, exendin-POEGMA_{10K}, OVA-PEG_{10K}, and OVA-POEGMA_{10K}. Exendin-PEG_{10K} and exendin-POEGMA_{10K} conjugates were synthesized, purified, and characterized. Briefly, azide functional, linear PEG was purchased from Creative PEGWorks. Azide functional POEGMA was synthesized by reacting EG3 (2.5 mmol; 565.4 μL), the catalytic complex (0.1 mmol TPMA and 0.01 mmol CuBr2; 62.5 μL), the polymerization initiator (62.5 μL in methanol; 0.01 mmol) in a mixture of methanol (1437.5 μL) and 100 mM NaCl (4432.8 μL) for 2 hours. Exendin-PEG_{10K} and exendin-POEGMA_{10K} conjugates were analyzed for Mₙ, M_{w}, and Ð using SEC-MALS.

OVA conjugates of PEG and POEGMA were synthesized via activated carbonate-amine conjugation. Nitrophenyl-carbonate (NPC) functional PEG was purchased from Creative PEGWorks. A hydroxyl functional POEGMA was synthesized by reacting EG3 (10 mmol; 2261.6 μL), the catalytic complex (0.08 mmol TPMA and 0.01 mmol CuBr₂; 100 μL), 2-Hydroxyethyl 2-bromoisobutyrate (Sigma) (200 μL in methanol; 0.04 mmol) in a mixture of methanol (5800 μL) and 100 mM NaCl (11.638 mL) for 1.5 hours. The resulting hydroxyl-functional POEGMA (5 mM in DCM) was reacted with p-nitrophenyl carbonate (100 mM in DCM) in the presence of pyridine (240 mM in DCM) for 16 hours to convert the hydroxyl end-group to an NPC. The resulting NPC-functional POEGMA was purified via filtration followed by diethyl ether precipitation. NPC functionalization yield was 79.7%, calculated by nuclear magnetic resonance (NMR) spectroscopy. NPC functional POEGMA and PEG were reacted with OVA (Invivogen; 2 mg ml⁻¹) at 10:1 ratio for 5 hours in 200 mM phosphate buffer at pH 8, yielding OVA-PEG_{10G} and OVA-POEGMA_{10K} conjugates. The resulting conjugates were purified by anion exchange chromatography to >98% purity using 20 mM sodium phosphate buffer at pH 8.6 with a NaCl gradient of 0-50%, desalted, and lyophilized. The conjugates were analyzed for M, M, and Ð using SEC-MALS.

Synthesis and characterization of drug coupled Luminex magnetic beads. Drugs—exendin, exendin-PEG_{10K}, exendin-POEGMA_{10K}, OVA, OVA-PEG_{10K}, and OVA-POEGMA_{10K}—were coupled to different sets of fluorescently barcoded MagPlex magnetic beads (Luminex) via carbodiimide chemistry by following the manufacturer's instructions with minor modifications The drug used in the coupling reactions was carefully titrated to ensure that the resulting bead sets had equal amounts of antigen.

To couple exendin (Santa Cruz Biotechnology), exendin-PEG_{10K}, and exendin-POEGMA_{10K}, beads were first amine-functionalized. The amine-modified beads were then conjugated to the exendin variants. Briefly, 12.5 million beads were rinsed with 0.5 ml of coupling buffer II (0.1 M MES; pH 6.0). Rinsed beads were incubated with 500 μL of adipic acid dihydrazide (ADH) (35 mg ml⁻¹) and 100 μL of EDC (200 mg ml⁻¹) for 1 hour. The resulting amine-modified beads were washed and resuspended in 200 μL of the coupling buffer II. 31.8 μL of exendin, 41.67 μL of exendin-PEG and 39.7 μL of exendin-POEGMA solutions (187.8 μM) and 25 μL of EDC (200 mg ml⁻¹) were added to amine-modified beads followed by bringing the final volume to 250 μL and incubation for 2 hours. The resulting drug-coupled beads were blocked overnight in assay buffer, which is 0.2% (w/v) I-Block protein-based blocking reagent (Thermo Scientific) in PBS (Hyclone). The next morning, they were washed three times, resuspended, and counted using a hemocytometer for final concentration.

In OVA, OVA-PEG_{10K}, and OVA-POEGMA_{10K} coupling reaction, 12.5 million beads were rinsed with 250 μL deionized water. Rinsed beads were resuspended in 50 μL activation buffer (0.1 M NaH₂PO₄; pH 6.2), followed by the addition of 25 μL of 100 mg ml⁻¹ N-hydroxy sulfosuccinimide (Sulfo-NHS; Thermo Scientific) and 1-Ethyl-3-[3-dimethylaminopropyl] carbodiimide hydrochloride (EDC; Thermo Scientific) and incubation for 20 minutes. Activated beads were washed with 1 mL coupling buffer (50 mM 2-(N-morpholino) ethanesulfonic acid (MES); pH 5.0) three times and resuspended in 500 μL of the coupling buffer. 93.7 μL of OVA, 106.6 μL of OVA-PEG_{10K}, and 105.6 μL of OVA-POEGMA_{10K} solutions (46.8 μM) were transferred, followed by bringing the total volume to 1 ml with the coupling buffer and incubation for 2 hours. The resulting drug-coupled beads were blocked overnight in the assay buffer, washed three times, resuspended, and counted using a hemocytometer. This protocol was also used to couple mouse IgG (Abcam; 2.5 μL; 2 mg ml⁻¹) and IgM (Bio-Rad; 6.25 μL; 2 mg ml⁻¹) as positive controls.

The resulting beads were characterized in drug conjugation using anti-drug antibodies at varied concentrations by following the manufacturer's instructions. Briefly, 50 μl of each set of beads (50,000 bead ml⁻¹ in the assay buffer) was transferred to a black, round-bottom 96-well-plate (Corning) Next, we prepared serial dilutions of mouse anti-OVA IgG (Abcam #17293), mouse anti-exendin IgG (Abcam #23407), mouse anti-PEG IgG (Abcam #195350), R-Phycoerythrin-conjugated goat anti-mouse IgG (Jackson Immunoresearch; 115-115-164), and biotinylated goat anti-mouse IgM (Jackson Immunoresearch; 115-065-075) in assay buffer. 50 μL of the resulting solutions were transferred to wells followed by incubation for 1 hour on an orbital shaker. After incubation, 96-well plate was placed on a magnetic ring stand (Invitrogen) and incubated for 60 seconds for separation to occur.

The supernatant was discarded, and wells were washed with 100 μl of the assay buffer twice. Drug-coupled beads were incubated with 100 μl of 5 μg ml⁻¹ R-Phycoerythrin-conjugated goat anti-mouse IgG (Jackson Immunoresearch; #115-115-164) for 30 minutes. Mouse IgG and IgM-coupled beads were incubated in assay buffer for 30 minutes. After incubation, 96-well-plate was placed on the magnetic ring stand and incubated for 60 seconds for separation to occur. The supernatant was discarded, and wells were washed with 100 μl of the assay buffer twice. Mouse IgM coupled beads were incubated with Streptavidin-R-Phycoerythrin Conjugate (SAPE) at 1.5 equivalent concentrations of biotinylated goat anti-mouse IgM used in that particular well for 30 minutes. 96-well-plate was placed on the magnetic ring stand and incubated for 60 seconds for separation to occur. The supernatant was discarded, and wells were washed with 100 μl of the assay buffer twice. Beads were solubilized in the assay buffer and analyzed using MAGPIX (Luminex).

The resulting bead sets had an equal amount of the same type of antigen. OVA, OVA-PEG₁₀ₖ, and OVA-POEGMA_{10K} bead sets had equal amounts of OVA, indicated by identical median fluorescence intensity (MFI) detected at varied mouse anti-OVA antibody concentrations. Similarly, exendin, exendin-PEG_{10K}, and exendin-POEGMA_{10K} bead sets had equal amounts of exendin. Importantly, we confirmed that exendin-PEG_{10K}- and OVA-PEG_{10K}-conjugated bead sets had equal amounts of PEG. We also confirmed the presence of mouse IgG and mouse IgM on beads, which were used as positive controls in multiplexed immunoassays.

### Optimization and Validation of the Luminex Multiplexed Immunoassay

The Luminex multiplexed immunoassay was optimized in its background, specificity, sensitivity, precision, and linearity. The optimized assay was validated by performing a spike-and-recovery experiment.

Background. The Limit of Blank (LoB) was defined as median fluorescence intensity (MFI) of singlet and multiplexed drug-coupled magnetic beads in assay buffer. Singlet LoB (SLoB) and multiplexed LoB (MLoB) were calculated by adding three standard deviations to the mean MFI in the assay buffer. The highest SLOB was 42 MFI, roughly corresponding to 0.26% of MFI detected with anti-drug antibodies, indicating that drug-coupled beads have a low fluorescence background. Importantly, SLOB and MLoB were not significantly different (P>0.99), indicating that multiplexing beads do not affect their fluorescence background.

Specificity. We tested if control antibodies (anti-exendin IgG, anti-OVA IgG, anti-PEG IgG, anti-mouse IgG, and anti-mouse IgM) showed any cross-reactivity to the drug-coupled beads by incubating singlet and multiplexed beads with a single type or multiple types of antibodies. Cross-reactivity of a bead set to a control antibody was calculated as the percent MFI signal of a true positive bead set and was less than 1% for all drug-coupled beads at 1 μg ml⁻¹ antibody concentration. This result indicated that the control reagents were of high specificity. Similarly, the assay buffer showed no cross-reactivity to anti-drug antibodies or drugs.

Sensitivity. The sensitivity of the immunoassay was assessed at varying concentrations of the control antibodies. The Limit of Detection (LoD) was defined as the minimum detectable control antibody dose for a particular drug-coupled bead and calculated for each bead set as mean antibody concentration plus three standard deviations. LoD was below 1.5 ng ml⁻¹ for all antigens, indicating that the assay was of high sensitivity. The lower limit of quantification (LLoQ) and upper limit of quantification (ULoQ) defined lower and upper boundaries of the assay's linear working range for each bead set.

Linearity. The assay's linearity was determined by assessing whether assay values were proportional to the analyte concentration. It was defined as the goodness of fit (R²) of at least four dilutions of plasma or control antibodies in the assay buffer. R² values were greater than 0.98 for all bead sets, indicating that working conditions remained in the assay's dynamic range.

Precision. Precision was determined by assessing the repeatability and the reproducibility of the assay and defined as intra-assay and inter-assay variability. Intra-assay variability (% CV) was calculated by dividing the standard deviation of anti-drug IgG concentration measured using drug-coupled beads (n=9) on the same plate by its mean. Inter-assay variability (% CV) was calculated by dividing the standard deviation of anti-drug IgG concentration measured using drug-coupled beads on three different plates (n=3) by its mean. Intra-assay and inter-assay variabilities were 6.57% and 9.57% for all antigens, indicating that the assay had high precision.

Validation. The optimized multiplexed immunoassay was validated by performing a spike-and-recovery experiment. Briefly, we spiked PBS-treated mouse plasma with 25 ng ml⁻¹ of exendin, ovalbumin, and PEG antibodies and calculated the percent drug recovered from the assay at varying dilutions. The % recovery was 105.4±9.6 in the assay buffer and 102±7.7 in the plasma, validating the assay performance.

### Design, Optimization, and Validation of a Cell-Based Neutralizing Antibody Assay

Assay Design. The presence of neutralizing antibodies (NAb) was tested in mice sera using the in vitro cell-based assay used to measure the activity of exendin variants with minor modifications. Briefly, the mice serum samples were incubated with exendin, Ex-PEG_{Mw}, or Ex-POEGMAₒₚₜ for 2 hours at room temperature. HEK293/CRE-Luc/GLP1R cells were then treated with the serum: drug mixtures (10% v/v) for 5 hours at a final concentration of the drugs' respective half-maximal effective concentration (EC50), followed by measuring luminescence. This assay allowed us to determine the binding antibodies' neutralizing ability, such that if there were NAbs present, they interacted with the drugs and blocked their binding to GLP1R, preventing cAMP induction to a decrease in luminescence.

Assay optimization. The cell-based neutralizing antibody assay was optimized in terms of matrix interference and sensitivity.

Matrix interference. The matrix interference was tested in terms of HEK293/CRE-Luc/GLP1R cells' ability to respond to a fixed concentration of exendin at varying dilutions of PBS-treated C₅₇BL/6J mice sera. We found that mice sera ≤5% (volume) did not significantly affect cell behavior. Therefore, the final serum volume was kept at 5% and constant across the assays.

Sensitivity. The sensitivity of the cell-based neutralizing antibody assay was tested using anti-exendin (NBP1-05179H; Novus Biologicals) and anti-PEG (ab195350; Abcam) antibodies as positive controls and anti-OVA antibody (ab17293; Abcam) as a negative control. Briefly, exendin, Ex-PEG_{Mw}, or Ex-POEGMAₒₚₜ were preincubated with varied concentrations of anti-exendin, anti-PEG, and anti-OVA antibodies, which were diluted in 5% PBS-treated C₅₇BL/6J mice sera, followed by treating HEK293/CRE-Luc/GLP1R cells and measuring luminescence. The luminescence signal deriving from each treatment group was represented as the percentage of the mean signal of cells treated without antibodies. Anti-exendin antibodies inhibited exendin, Ex-PEG_{Mw}, or Ex-POEGMAₒₚₜ, while anti-OVA antibodies did not have any effect on the drugs' activity. Anti-PEG antibodies only inhibited Ex-PEG_{Mw} and did not affect the activity of exendin and Ex-POEGMAₒₚₜ. The assay sensitivity was given as the half-maximal inhibitory concentration (IC₅₀) of the antibodies. The assay was more sensitive for exendin (7.2±1.1 nM) than Ex-PEG_{Mw} (26.9±5.3 nM) and Ex-POEGMAₒₚₜ (23.5±3.1 nM) due to the lower concentration of exendin (0.15 nM) used in the assay than Ex-PEG_{Mw} (2.7 nM) and Ex-POEGMAₒₚₜ (2.8 nM). The molar equivalent of anti-exendin antibody needed to inhibit Ex-PEG_{Mw} (10.0±2.0) and Ex-POEGMAₒₚₜ (8.4±1.1) half-maximally was higher than exendin (48±7.3) due to the steric hindrance imparted on exendin by conjugated PEG and POEGMA. The assay sensitivity for anti-PEG antibodies was 28.3±3.5 nM, which corresponded to the 10.5±1.3 molar equivalent of Ex-PEG_{Mw}.

### Example 3

### Injectable and Depot Forming POEGMA Library

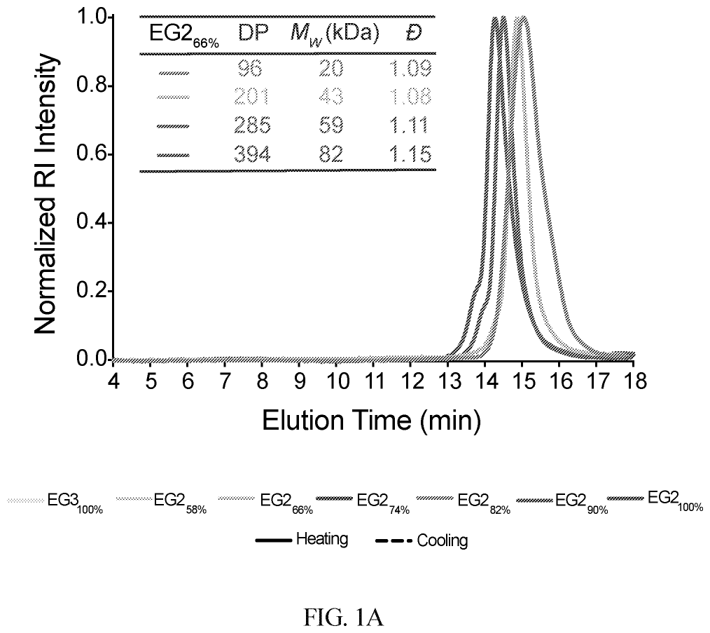

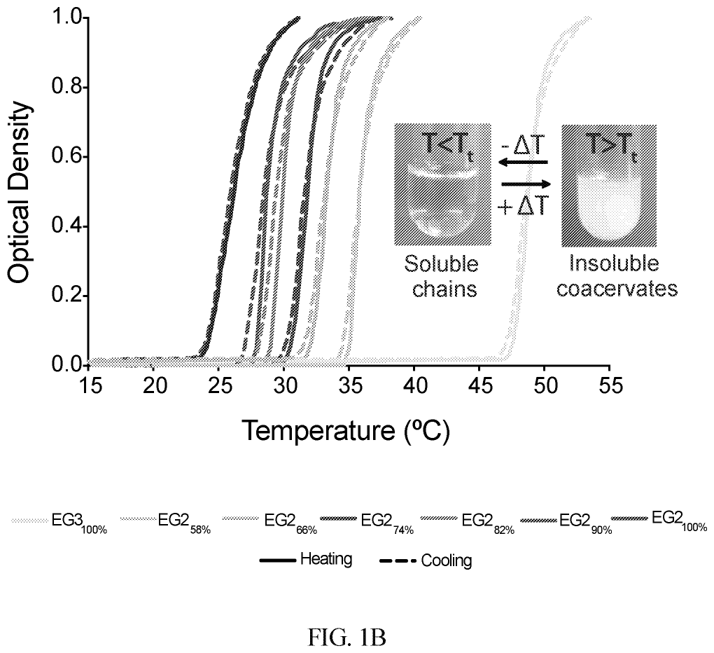

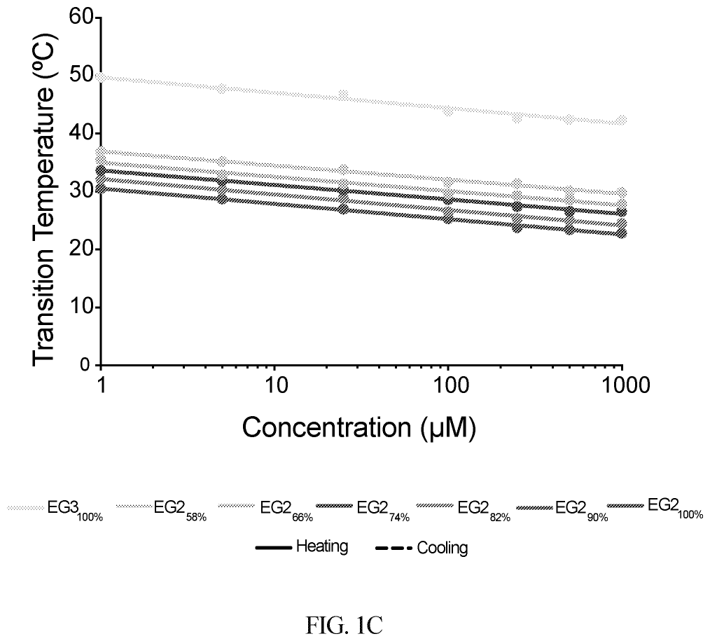

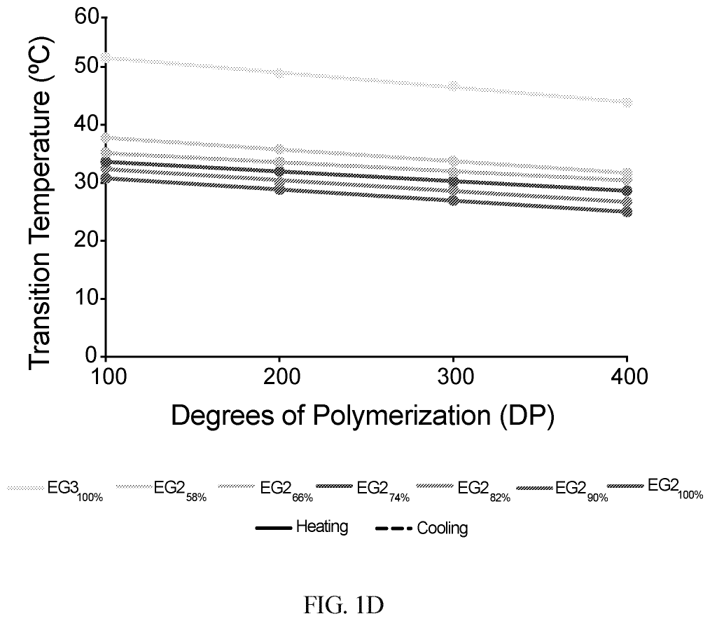

POEGMA exhibits a lower critical solution temperature (LCST) phase behavior, allowing it to phase transition between soluble and insoluble forms in a temperature- and concentration-dependent manner. We exploited this beneficial feature to create a drug depot under the skin and achieve sustained release of the drug from the depot into the circulation. We identified POEGMAs that would be suitable as an injectable depot by creating a set of POEGMAs with azide-end groups that phase transition between room temperature (25° C.) and subcutaneous (s.c.) temperature of mice (34° C.) using activator-regenerated by electron transfer atom transfer radical polymerization (ARGET-ATRP). We restricted the EG side-chain length to ≤3 because previous studies indicated that the side-chain length of >3 had some degree of binding with PEG antibodies. We also fortuitously found that the phase transition temperature (Tₜ) of POEGMA was sensitive to the OEG side chain length, with shorter side-chains having a lower Tₜ due to the decreased hydrogen bonding. The Tₜ of a homopolymer POEGMA with 3EG-long side chains (EG3) was too high to form an s.c. depot, while Tₜ of an EG2 homopolymer was too low, preventing it from being injected in the soluble form at room temperature. Hence, we synthesized a set of copolymers using EG2 and EG3 monomers at various ratios in which the molar percentage of EG2 ranged from 58-100%, determined by Nuclear Magnetic Resonance (NMR) spectroscopy. We also synthesized an EG3 POEGMA homopolymer to use as a non-depot forming—soluble—control. The monomer composition of POEGMA was defined as the percentage of EG2 (or EG3) monomer content in the total polymer. We used the nomenclature of EGX_{%}, where X is the length of the EG chain (2 or 3), and the subscript % is the percentage of the monomer in the total polymer. The polymers were monodisperse as measured by gel permeation chromatography-multi-angle light scattering (GPC-MALS) with a polydispersity (Ð) of <1.2 (FIG. 1A). Their degree of polymerization (DP) ranged from 100 to 400, translating to a weight-averaged molecular weight (M_{w}) range of 20-80 kDa. This M_{w} range should clear by the kidney at varying rates, thereby allowing control over PK. Each polymer's monomer composition, M_{w}, number-averaged molecular weight (Mₙ), Ð, DP, hydrodynamic size (Rₕ), and Tₜ were summarized in Table 1.

TABLE 1
Summary of POEGMA library characterization. The monomer composition of
POEGMAs was defined as the percentage of EG2 (or EG3) content in the copolymer and
derived from the nuclear magnetic resonance (NMR) spectra. Degrees of polymerization (DP),
number-averaged molecular weight (Mₙ), weight-averaged molecular weight (M_{w}), and
polydispersity (Ð) values were determined from gel permeation chromatography-multi-angle
light scattering (GPC-MALS). Hydrodynamic size (Rₕ) was calculated from dynamic light
scattering data. Transition temperature (Tₜ) values were derived from UV-vis spectrophotometry
curves given in FIG. 1b-d.

| | | EG2% | | | | | | |
|---|---|---|---|---|---|---|---|---|
| | POEGMA | by | | | | | | |
| Group | ID | NMR | DP | M_{w} | Mₙ | Ð(M_{w}/Mₙ) | Rₕ(nm) | Tt(25 μM; ° C.) |
| EG3_{100%} | P1 | 0 | 105 | 24.7 | 22.7 | 1.079 | 3.4 ± 0.6 | 51.6 |
| | P2 | 0 | 194 | 45.3 | 40.3 | 1.123 | 4.6 ± 1.0 | 48.9 |
| | P3 | 0 | 314 | 73.2 | 68.0 | 1.075 | 6.5 ± 1.4 | 46.7 |
| | P4 | 0 | 386 | 90.0 | 84.7 | 1.063 | 6.7 ± 1.3 | 43.9 |
| EG_{258%} | P5 | 58.4 | 95 | 19.9 | 18.3 | 1.088 | 3.0 ± 0.5 | 37.8 |
| | P6 | 58.5 | 206 | 42.7 | 39.6 | 1.078 | 3.4 ± 0.4 | 35.8 |
| | P7 | 59.0 | 285 | 59.1 | 53.1 | 1.113 | 4.8 ± 1.2 | 33.8 |
| | P8 | 56.5 | 394 | 82.0 | 71.6 | 1.146 | 5.1 ± 0.4 | 31.7 |
| EG2_{66%} | P9 | 65.5 | 96 | 19.8 | 18.1 | 1.092 | 2.9 ± 0.3 | 35.2 |
| | P10 | 65.0 | 201 | 41.2 | 38.6 | 1.066 | 3.2 ± 0.4 | 33.6 |
| | P11 | 68.8 | 298 | 60.5 | 55.3 | 1.095 | 3.9 ± 0.3 | 31.8 |
| | P12 | 65.0 | 389 | 79.5 | 69.4 | 1.145 | 5.3 ± 0.7 | 30.6 |
| EG2_{74%} | P13 | 72.0 | 104 | 21.1 | 19.3 | 1.091 | 3.1 ± 0.4 | 33.7 |
| | P14 | 75.5 | 208 | 41.6 | 38.3 | 1.086 | 3.5 ± 0.3 | 32.0 |
| | P15 | 76.5 | 313 | 62.5 | 55.7 | 1.122 | 4.6 ± 0.8 | 30.3 |
| | P16 | 73.8 | 420 | 84.2 | 72.2 | 1.166 | 5.7 ± 1.1 | 28.7 |
| EG2_{82%} | P17 | 82.3 | 105 | 20.8 | 19.0 | 1.097 | 3.1 ± 0.7 | 32.6 |
| | P18 | 82.0 | 195 | 38.4 | 34.9 | 1.099 | 3.8 ± 0.5 | 30.1 |
| | P19 | 83.0 | 289 | 56.8 | 52.8 | 1.075 | 4.3 ± 0.5 | 28.8 |
| | P20 | 82.3 | 404 | 79.4 | 67.7 | 1.173 | 5.9 ± 1.3 | 26.7 |
| EG2_{90%} | P21 | 89.6 | 103 | 20.1 | 18.2 | 1.102 | 3.0 ± 0.5 | 30.8 |
| | P22 | 92.0 | 210 | 40.6 | 35.3 | 1.151 | 3.5 ± 0.3 | 28.9 |
| | P23 | 90.0 | 315 | 61.0 | 55.2 | 1.105 | 4.1 ± 0.5 | 27.0 |
| | P24 | 90.0 | 405 | 78.2 | 66.2 | 1.182 | 4.8 ± 0.4 | 25.0 |
| EG2_{100%} | P25 | 100 | 190 | 36.0 | 32.3 | 1.116 | 3.1 ± 0.5 | 25.8 |

All polymers showed a sharp and thermally reversible phase behavior with no hysteresis, as seen by the sharp increase in optical density as the temperature was increased. The optical density decreased as the temperature was decreased below the Tₜ (FIG. 1B). The EG3_{100%} POEGMA had a Tₜ of ˜48° C. at 25 μM, confirming that it cannot form an s.c. depot. The Tₜ of the POEGMA copolymers was a function of the EG2 content, as the Tₜ decreased with the increasing molar ratio of the more hydrophobic EG2 monomer. All copolymers phase transitioned between 25 and 34° C., suggesting that all copolymers were potential depot-forming constructs. The Tₜ of POEGMA also showed an inverse concentration dependence. The Tₜ increased with POEGMA dilution (FIG. 1C), suggesting that sustained release of a POEGMA conjugate from the s.c. depot should be possible in response to continuous dilution at the periphery of the depot. The Tₜ of POEGMA was also a function of M_{w}, with a decrease in Tₜ with increasing DP, thus M_{w} (FIG. ID), possibly due to lower degrees of freedom at higher M_{w}. These data clearly showed that key molecular variables controlling phase behavior of POEGMA depots were Tₜ and M_{w}.

### Example 4

### Exendin-POEGMA conjugate

We identified an exendin-POEGMA conjugate that maximized fed blood glucose control in diabetic mice by systematically tuning its Tₜ and M_{w}. To identify an exendin-POEGMA conjugate with an advantageous Tₜ, we synthesized conjugates with varying Tₜ using POEGMAs at varied monomer compositions (Table 2; index Tₜ). Conventional conjugation methods typically provide limited control over the conjugation site and stoichiometry, resulting in a heterogeneous mixture of conjugates with non-uniform PK and pharmacodynamics (PD). To circumvent this issue, we conjugated POEGMA to the C-terminus of exendin using bio-orthogonal click chemistry as its N-terminus is important to its function. We first attached a bio-orthogonal triglycine dibenzocyclooctyne (DBCO) group to the C-terminus of exendin via sortase A-mediated native peptide ligation, yielding exendin-DBCO. DBCO was chosen because it readily reacts with the azide end-group in POEGMA and does not react with any other chemical groups in exendin, yielding a stoichiometry of 1:1.

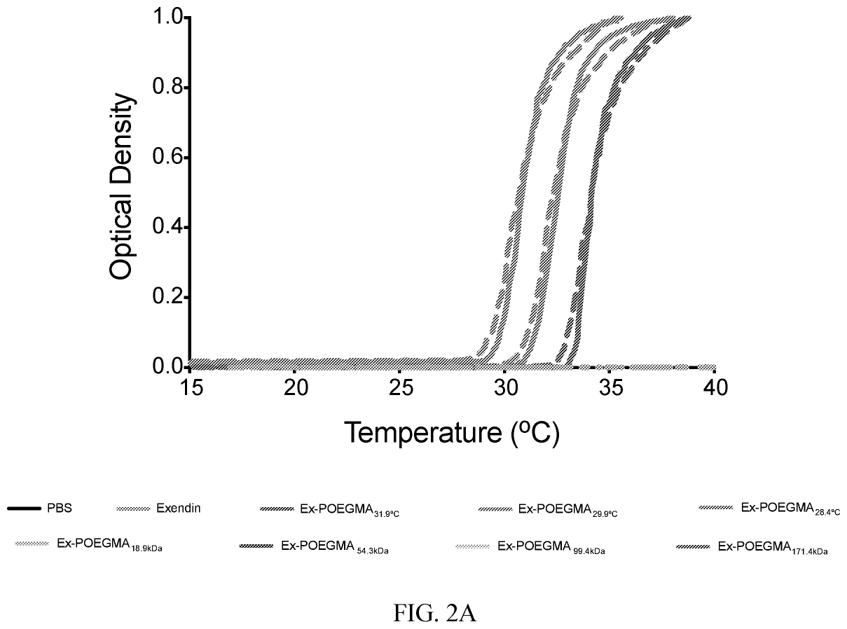

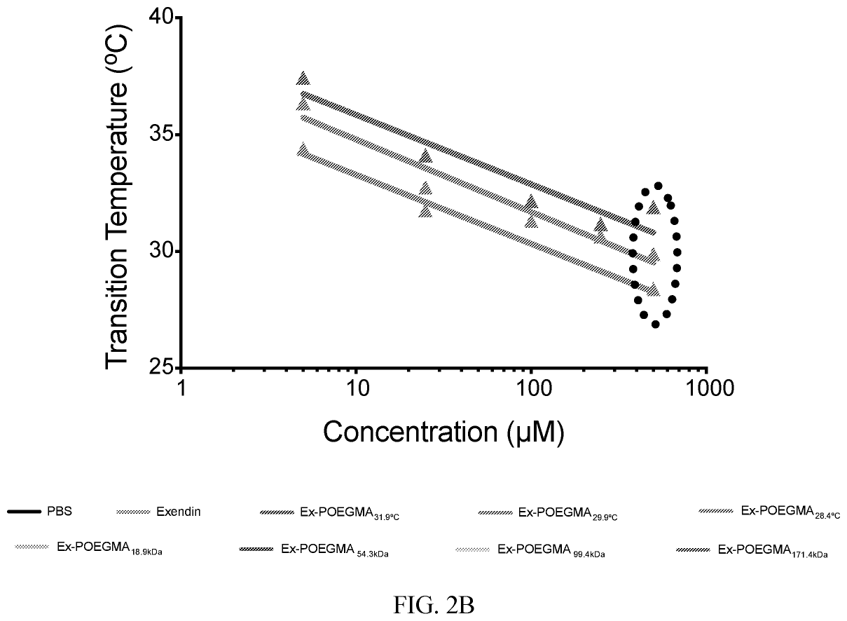

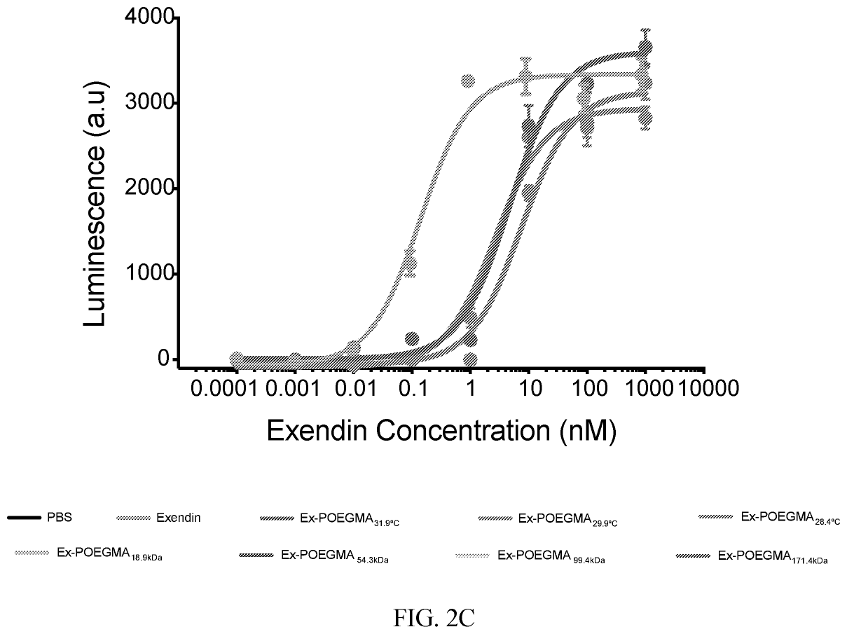

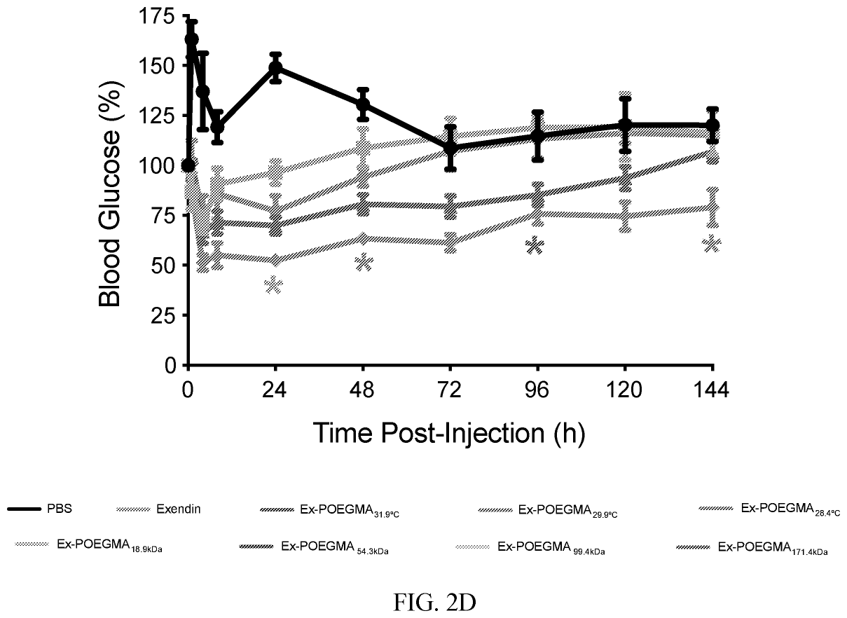

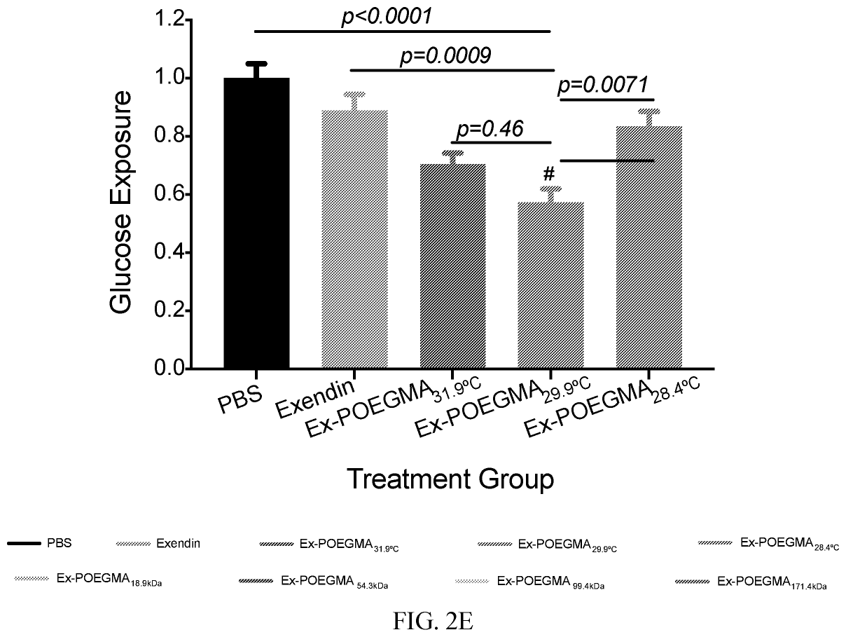

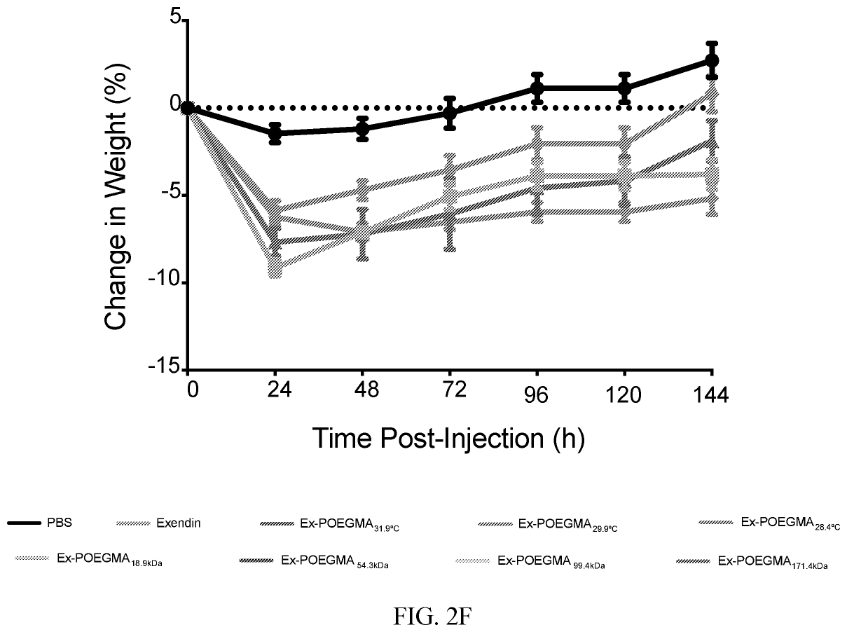

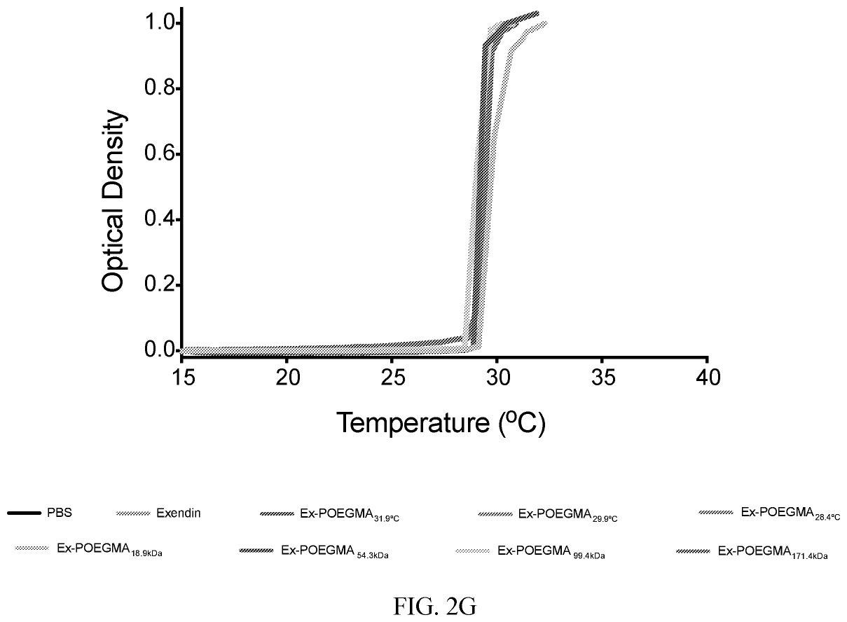

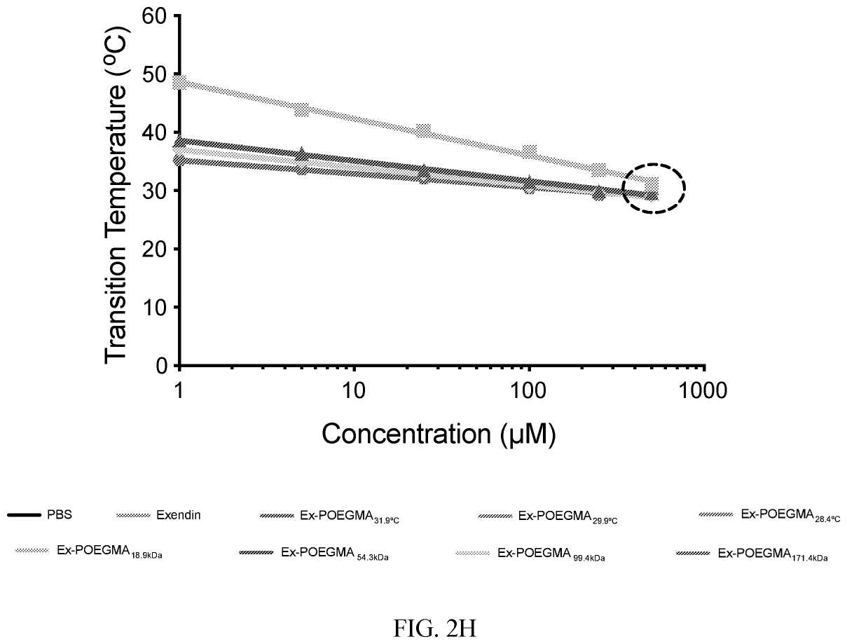

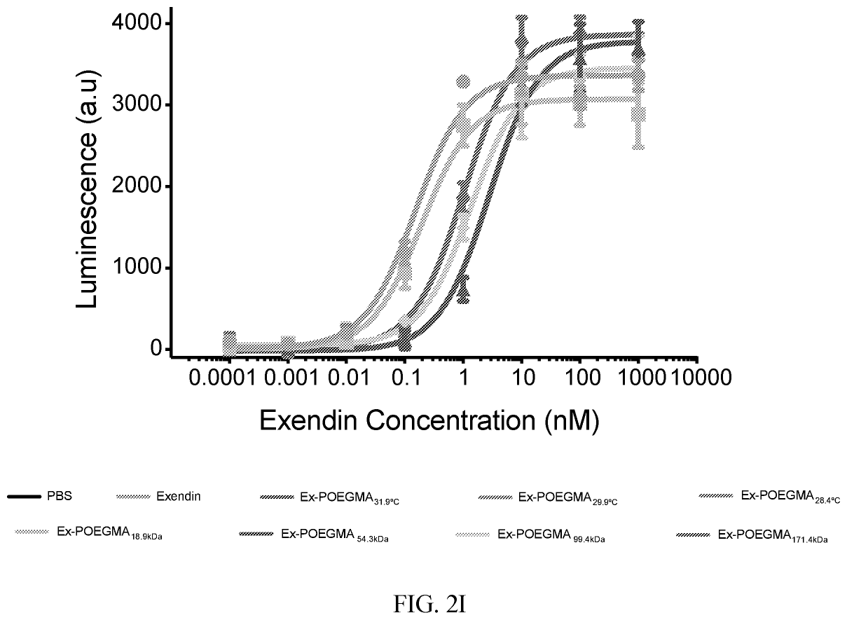

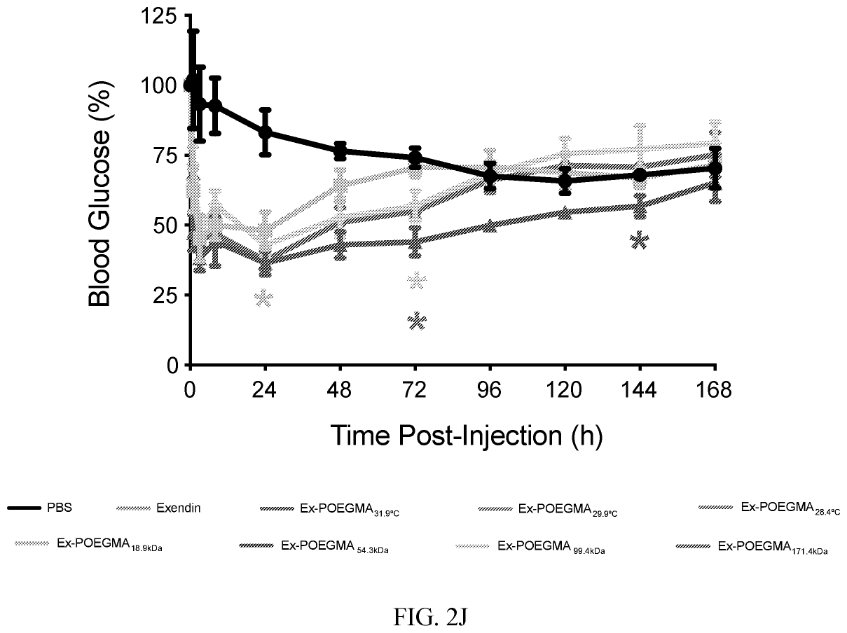

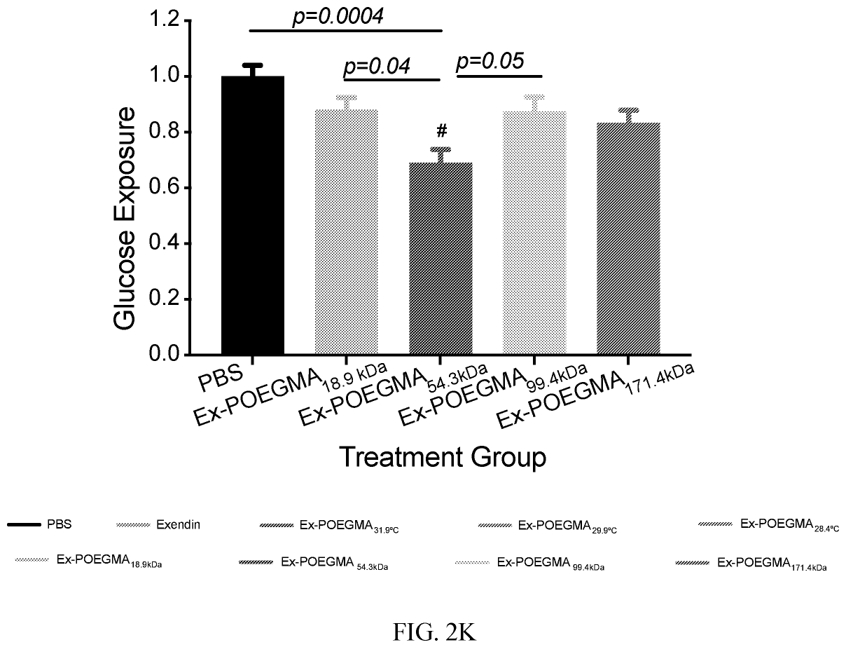

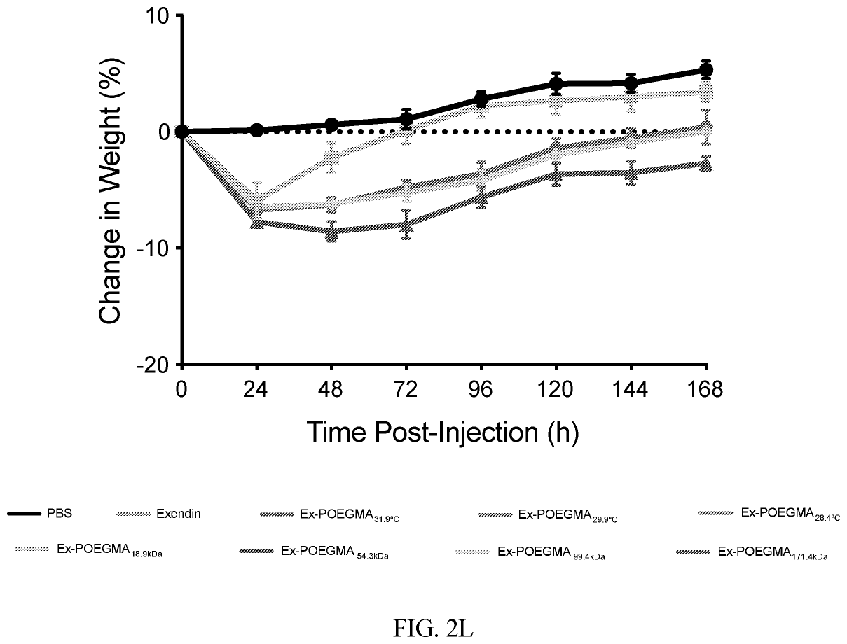

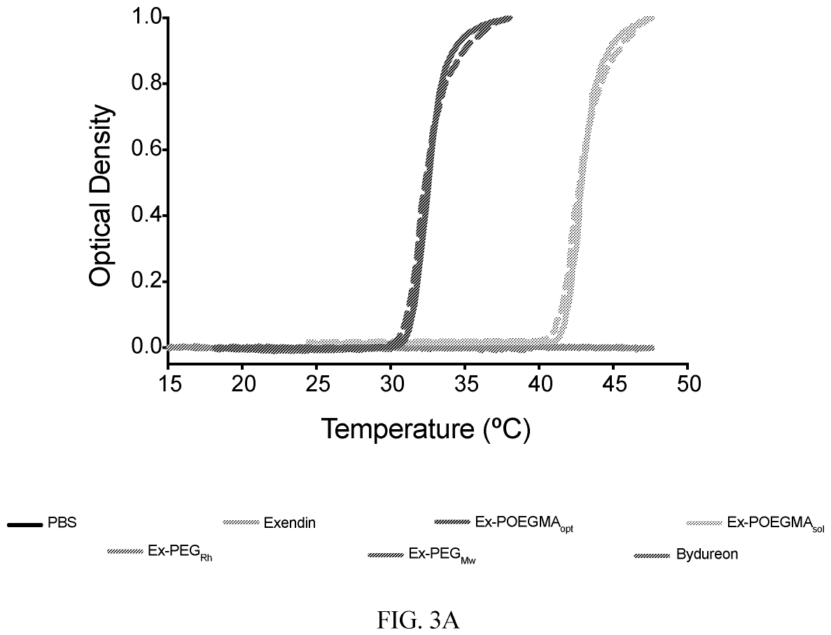

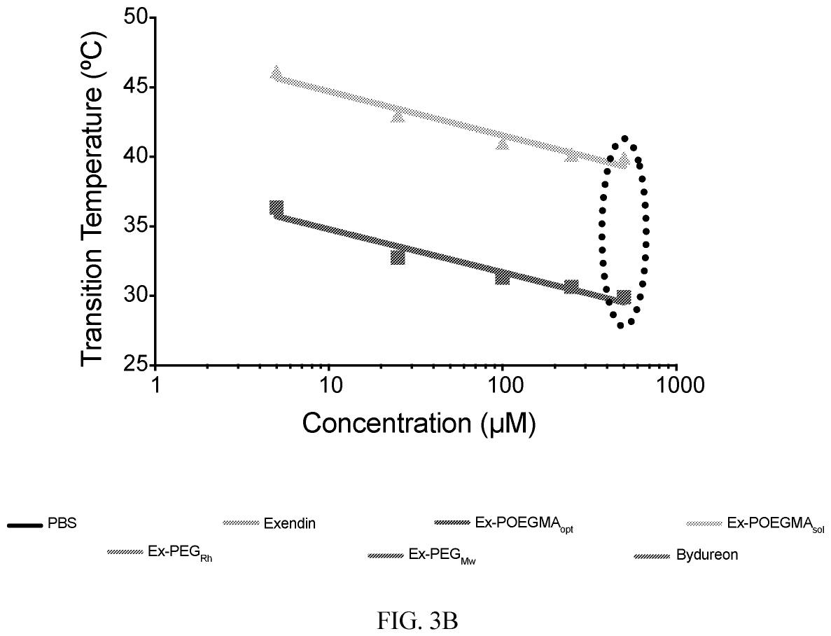

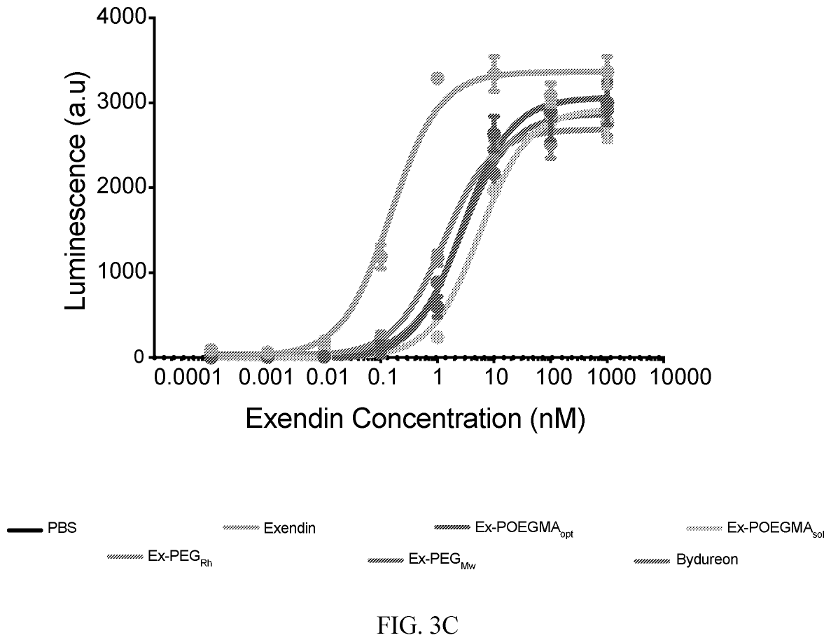

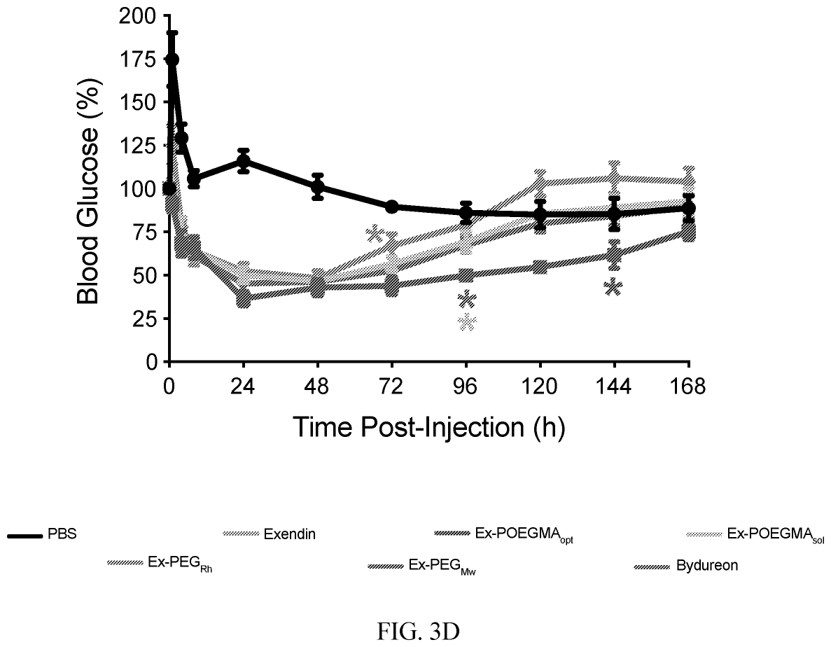

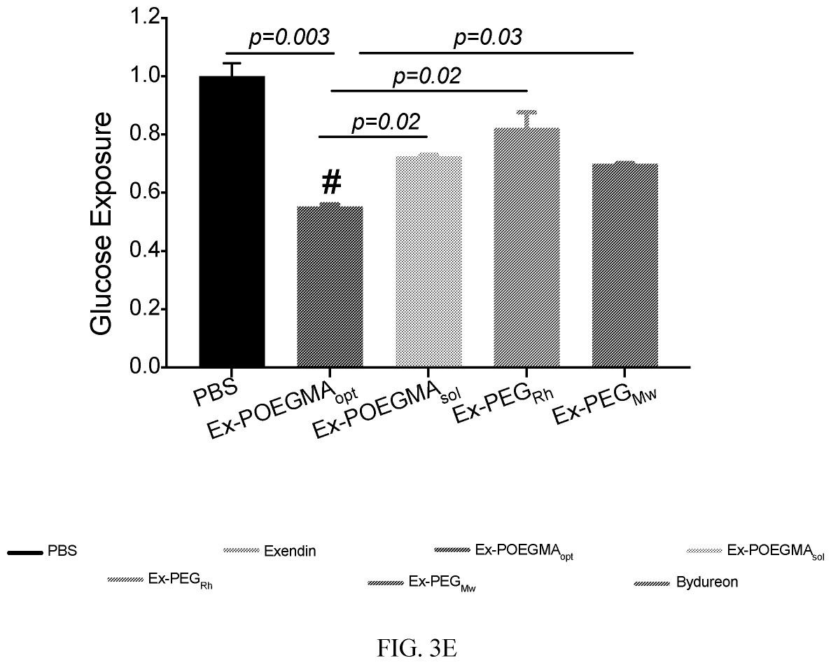

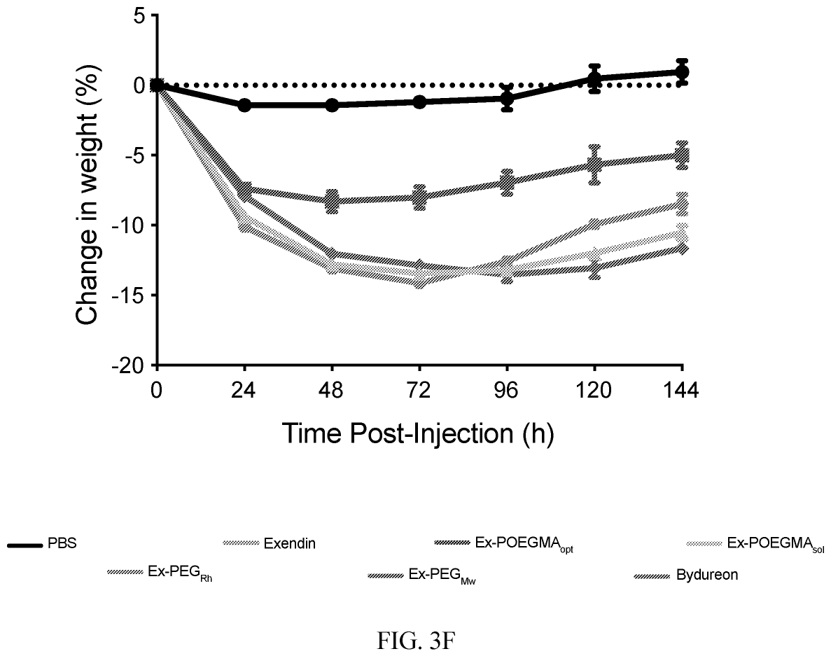

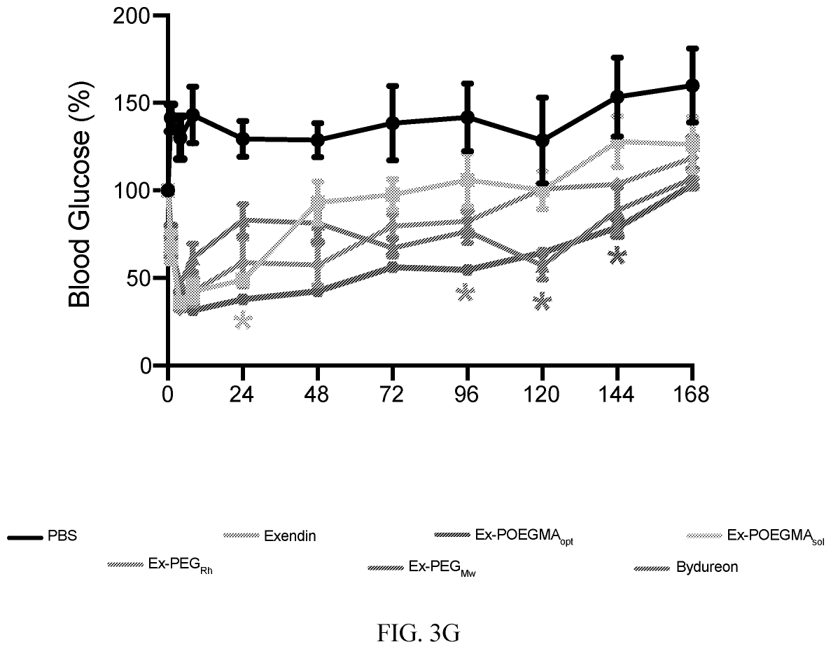

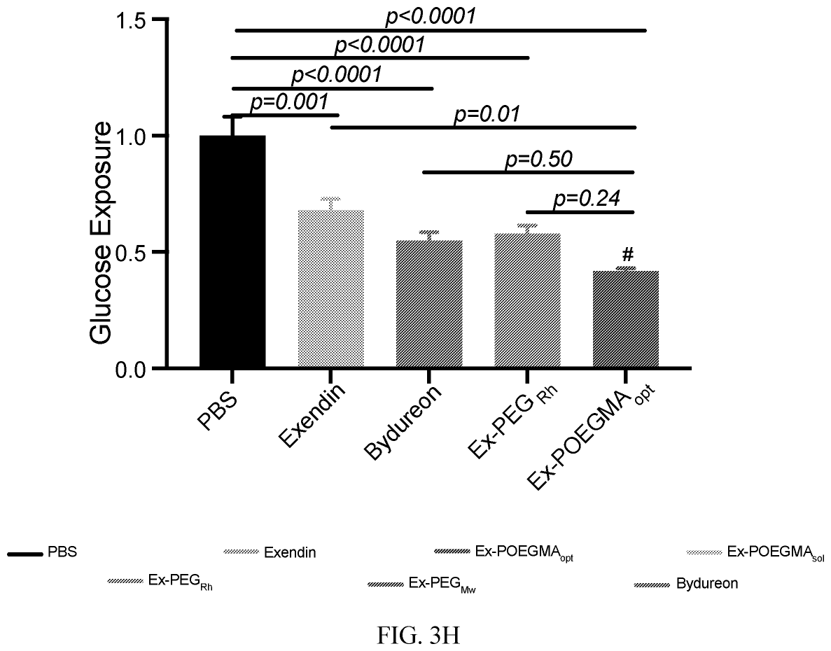

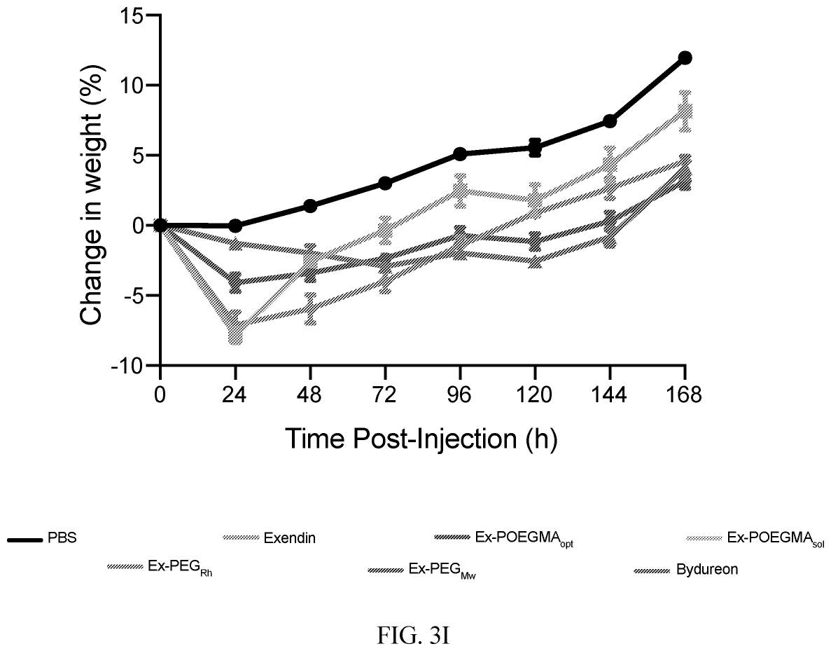

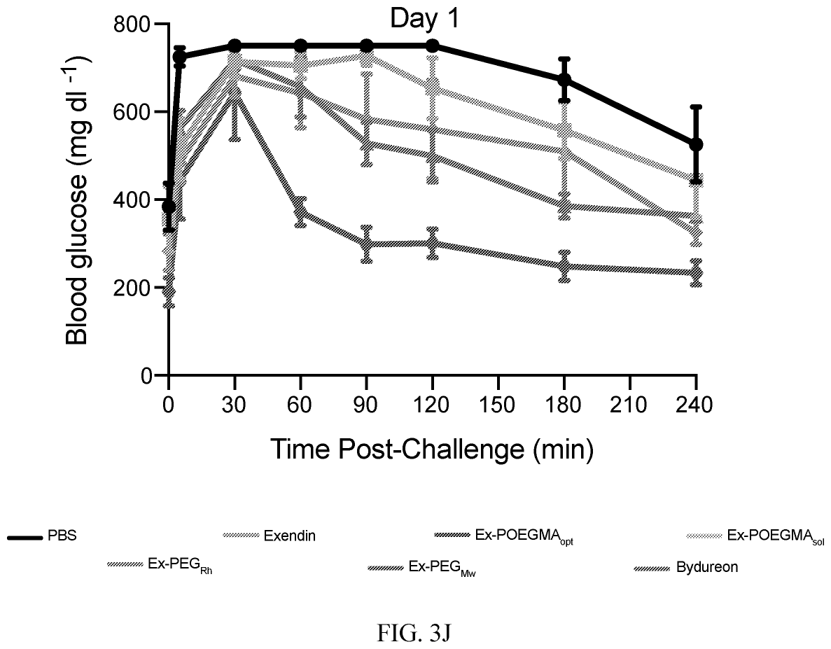

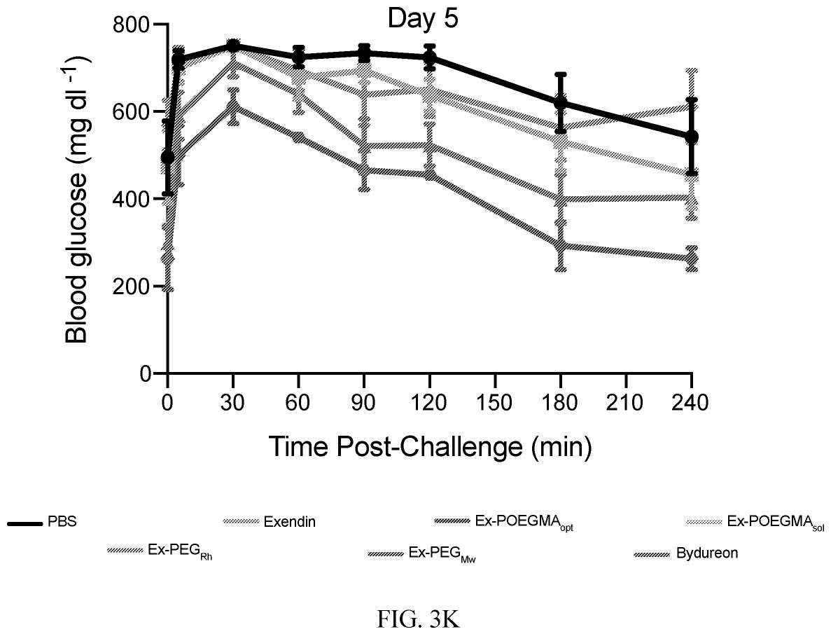

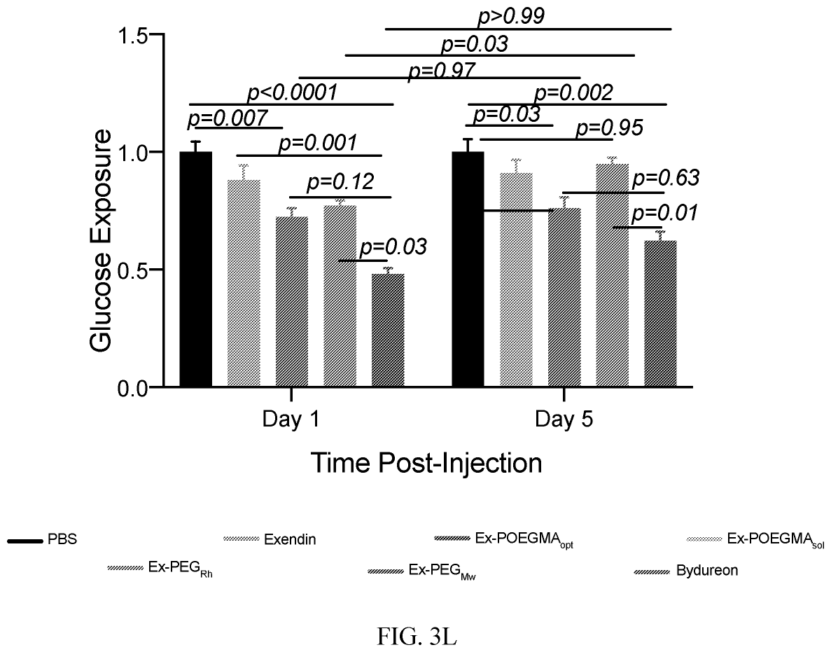

TABLE 2
Characterization of exendin variants. EG2% was calculated using NMR spectroscopy.
Mₙ, M_{w}, and Ð values were determined by SEC-MALS. Rₕ was measured by dynamic light
scattering (DLS) (n = 10). DP was calculated by dividing polymer M_{w} into its monomeric M_{w}.
The EC₅₀ values exendin variants were derived from the cAMP response curves given in FIG. 2C,
FIG. 2I, and FIG. 3C (n = 10). Data were reported as mean ± standard error of the mean (SEM).
Tₜ was measured at 500 μM. *Calculated from the amino acid sequence. †Default value due to
the monodisperse nature of the peptide.

| Compound | EG2% | DP | Tₜ(° C.) | M_{w}(kDA) | Ð(M_{w}/Mₙ) | Rₕ(nm) | EC₅₀(nM) |
|---|---|---|---|---|---|---|---|
| Exendin | N/A | N/A | N/A | 4.2* | 100† | 2.2 ± 0.1 | 1.14 ± 0.01 |
| Index: Tₜ | | | | | | | |
| Ex- | 48.5 | 247 | 31.9 | 57.7 | 1.15 | 4.8 ± 0.7 | 4.7 ± 0.8 |
| POEGMA_{31.9° C.} | | | | | | | |
| Ex- | 60.5 | 253 | 29.9 | 57.6 | 1.15 | 4.4 ± 0.9 | 3.0 ± 0.7 |
| POEGMA_{29.9° C.} | | | | | | | |
| Ex- | 68.8 | 254 | 28.4 | 56.9 | 1.13 | 4.5 ± 0.7 | 4.5 ± 1.1 |
| POEGMA_{28.4° C.} | | | | | | | |
| Index: Mw | | | | | | | |
| Ex- | 100 | 71 | 32.3 | 18.9 | 1.03 | 2.6 ± 0.9 | 1.2 ± 0.4 |
| POEGMA_{18.9kDA} | | | | | | | |
| Ex- | 62.5 | 239 | 29.6 | 54.3 | 1.14 | 4.2 ± 0.5 | 2.8 ± 0.7 |
| POEGMA_{54.3kDA} | | | | | | | |
| Ex- | 56.8 | 454 | 29.0 | 99.4 | 1.13 | 4.5 ± 1.1 | 1.4 ± 0.3 |
| POEGMA_{99.4kDA} | | | | | | | |
| Ex- | 50.8 | 792 | 29.4 | 171.4 | 1.16 | 5.1 ± 1.0 | 1.8 ± 0.2 |
| POEGMA_{171.4kDA} | | | | | | | |
| Index: Polymer type | | | | | | | |
| Ex- | 62.3 | 241 | 29.9 | 54.7 | 1.09 | 4.1 ± 0.5 | 2.8 ± 0.7 |
| POEGMAₒₚₜ | | | | | | | |
| Ex- | N/A | 221 | 40.1 | 56.6 | 1.09 | 4.3 ± 0.3 | 3.6 ± 0.9 |
| POEGMAₛₒₗ | | | | | | | |
| Ex-PEG_{Rh} | N/A | 454 | N/A | 27.7 | 1.07 | 4.4 ± 1.1 | 1.3 ± 0.2 |
| Ex-PEG_{Mw} | N/A | 909 | N/A | 46.3 | 1.10 | 5.2 ± 1.0 | 2.7 ± 0.5 |

The exendin-POEGMA conjugates had a constant M_{w} of ˜57 kDa, previously shown as the M_{w} that maximized the PK of a soluble exendin-POEGMA conjugate (Table 1). They were monodisperse and had similar Rₕ (Table 1). They showed reversible phase transition (FIG. 2A). The Tₜ of the conjugates were between 28-32° C., allowing the conjugates to remain in solution at room temperature and transition to insoluble coacervates when injected s.c, as tested at an injection concentration of 500 μM (see circled data in FIG. 2B). The Tₜ of the conjugates also showed an inverse concentration-dependence (FIG. 2B), indicating that they could be released from the depot into the blood in response to dilution at the depot's margins. The conjugates with lower Tₜ had higher, more hydrophobic EG2 content (Table 1). Finally, the conjugates were tested for their ability to activate exendin's endogenous receptor, termed glucagon-like peptide 1 receptor (GLP1R), in an in vitro cell-based assay using unconjugated exendin and PBS as controls. The conjugates showed high potency in activating GLP1R (FIG. 2C) but had a lower half-maximal effective concentration (EC₅₀) than exendin due to the steric hindrance imparted by the conjugated POEGMA. No significant difference was observed among the EC₅₀ of depot-forming conjugates.

We next examined the fed blood glucose control provided by the exendin-POEGMA conjugates by s.c. administering them into 11-week-old diet-induced obese (DIO) C₅₇BL/6J mice. The equivalent dose of exendin and PBS injection volume were used as positive and negative controls, respectively. Mice treated with exendin and the conjugates had lower blood glucose levels (FIG. 2D) and body weight (FIG. 2F) compared to the PBS-treated group. Exendin provided shorter control than the conjugates due to its short half-life. The most hydrophobic conjugate tested—Ex-POEGMA_{28.4}—resulted in only modest control, possibly because it released only a small amount of drug. The Ex-POEGMA_{29.9} conjugate outperformed others by providing six days of glucose control and had the lowest area under the curve (AUC) for glucose exposure (FIG. 2E), which accounts for both magnitude and duration of blood glucose reduction.

Having identified an exendin-POEGMA conjugate with an advantageous Tₜ, we next identified a M_{w}. Our strategy was to synthesize exendin-POEGMA conjugates with a Tₜ of ˜30° C. at varying M_{w} (Table 1; Index M_{w})—18.9 kDa, 54.3 kDa, 99.3 kDa, and 171.4 kDa—that should be cleared by the kidney at varying rates, thereby allowing control over the PK. We achieved this by titrating POEGMAs in terms of their monomer composition. The conjugates with lower M_{w} had higher, more hydrophobic EG2 content in POEGMA. The resulting exendin-POEGMA conjugates had varied Rₕ and reversibly phase transitioned (FIG. 2G). The conjugates showed slightly different inverse concentration dependence due to varying mass % of POEGMA in each conjugate, indicated by varying slopes of log(concentration) versus Tₜ plots (FIG. 2H), suggesting differences in drug release profiles. Importantly, they intersected at 500 μM with a Tₜ of ˜30° C. (29-32.3° C.) (see circled data in FIG. 2H). This concentration, which yielded near-constant Tₜ across conjugates, was chosen as the injection concentration for all in vivo studies. No significant correlation was observed between the EC₅₀ and M_{w} of conjugates (FIG. 21).

The resulting conjugates were next administrated into 11-week-old DIO mice (n=6) using PBS as a control. Mice treated with the conjugates had lower blood glucose (FIG. 2J) and body weight (FIG. 2L) than the PBS-treated group. The conjugate with the lowest M_{w} tested—Ex-POEGMA_{18.9}—controlled blood glucose only one day and had a significantly higher glucose exposure (FIG. 2K) than all other treatment groups. This data suggests that Ex-POEGMA_{18.9kDa} was cleared from the circulation earlier than the rest of the conjugates because it was smaller (Rₕ˜2.6 vs. ˜4.5 nm) than the renal excretion threshold, defined as the size of serum albumin (˜3.6 nm). The higher M_{w} exendin-POEGMA conjugates differed in glucose control duration due to their varying drug release profiles affecting the drug concentration in circulation. Ex-POEGMA_{99.4} and Ex-POEGMA_{171.4} provided modest glucose control for three days, while Ex-POEGMA_{54.3} outperformed them with six days of glucose control and had the lowest AUC for glucose exposure. The Tₜ- and M_{w}-optimal conjugate was subsequently referred to as exendin-POEGMAₒₚₜ (Ex-POEGMAₒₚₜ) and dose optimized in a dose-escalation study, where the optimal injection dose was determined as 1000 nmol kg⁻¹.

### Example 5

### Short-Term Efficacy

We next investigated the short-term efficacy of Ex-POEGMAₒₚₜ compared to its soluble POEGMA and PEG counterparts. We synthesized the soluble counterpart of Ex-POEGMAₒₚₜ—termed Ex-POEGMAₛₒₗ—using POEGMA consisting of only EG3 monomers at the same M_{w}. In preliminary studies, we found that linear PEG had a much larger fingerprint than hyperbranched POEGMA, resulting in conjugates with a much larger Rₕ at the identical M_{w}. Because this difference could complicate side-by-side efficacy comparison of the conjugates by affecting their kidney clearance rates, we synthesized both M_{w}-matched and Rₕ-matched exendin-PEG conjugates—termed Ex-PEG_{Mw} and Ex-PEG_{Rh} (Table 1). Ex-POEGMAₒₚₜ reversibly phase transitioned below body temperature (FIG. 3A), allowing it to remain as a solution in a syringe at room temperature but to transition to insoluble coacervates when injected s.c, as tested at a concentration of 500 μM (see circled data in FIG. 3B). In contrast, Ex-POEGMAₛₒₗ phase-transitioned well above the body temperature at all concentrations, indicating that it cannot form a depot. Neither Ex-PEG_{Mw} nor Ex-PEG_{Rh} showed phase behavior (FIG. 3A). The conjugates showed no difference in terms of their EC50 in activating GLP1R (FIG. 3C).

The conjugates were next s.c. administered into 11-week-old DIO mice (n=6) at the equivalent, optimal dose using PBS as a control. Mice treated with the conjugates had lower fed blood glucose levels than the control (FIG. 3D). Ex-PEG_{Rh} and Ex-PEG_{Mw} controlled fed blood glucose for three and four days, respectively (FIG. 3D). This difference was attributed to the larger size, thereby slower clearance, of Ex-PEG_{Mw}. Ex-POEGMAₛₒₗ provided modest blood glucose control for four days. Ex-POEGMAₒₚₜ outperformed its soluble POEGMA and PEG counterparts by providing six days of fed blood glucose control and the lowest glucose exposure (FIG. 3E), indicating the efficacy benefits provided by sustained release. All soluble conjugates resulted in a much more significant weight loss than Ex-POEGMAₒₚₜ (FIG. 3F), possibly because a sudden increase in their concentration in circulation induced nausea and thereby weight loss, a common side-effect of exendin.

Motivated by these results, we further tested the short-term efficacy of Ex-POEGMAₒₚₜ in db/db mice that display the most severe form of T2D in mice. db/db mice carry mutant leptin receptor gene and closely mimic T2D development in humans because leptin regulates appetite and satiety In this experiment, we also compared Ex-POEGMAₒₚₜ to Bydureon, a once-weekly administrated clinical sustained-release exendin formulation developed by encapsulating exendin into poly-lactic-co-glycolic acid (PLGA) microspheres. All treatment groups (n=5) resulted in lower glucose levels than the control (FIG. 3G). Exendin-POEGMAₒₚₜ stood out by lowering fed blood glucose for six days. A clear trend in glucose exposure (FIG. 3H) was observed, with Ex-POEGMAₒₚₜ having the lowest AUC. Soluble Ex-PEG_{Rh} induced a more significant weight loss than sustained-release formulations Ex-POEGMAₒₚₜ and Bydureon (FIG. 3I), consistent with the earlier results.

To support these results, we next tested if Ex-POEGMAₒₚₜ provided glycemic regulation by performing an intraperitoneal (i.p) glucose tolerance test (IPGTT) on days 1, 3, and 5 post-injection of the treatments into db/db mice. Exendin-treated mice could not tolerate the glucose challenge, indicated by hyperglycemia, due to its short half-life (FIGS. 3J-L). There was a clear trend in glucose exposure on days 1 and 3, with Ex-POEGMAₒₚₜ regulating blood glucose more efficiently than Bydureon. Ex-PEG_{Rh} lost its glycemic regulation ability by Day 3 due to its faster clearance. Notably, Ex-POEGMAₒₚₜ showed superior glycemic regulation to Ex-PEG_{Rh} throughout the study by providing the lowest glucose exposure among the treatments. This difference was also observed when the experiment was repeated using DIO mice, although to a lesser degree, possibly due to the milder display of T2D allowing lower circulating Ex-PEG_{Rh} concentrations to show therapeutic effect. The time of the test did not affect glucose exposure in the Ex-POEGMAₒₚₜ treatment group (p>0.99) (FIG. 3L), indicating that Ex-POEGMAopt maintained its superior glycemic regulation ability throughout the study.

### Example 6

### Pharmacokinetics

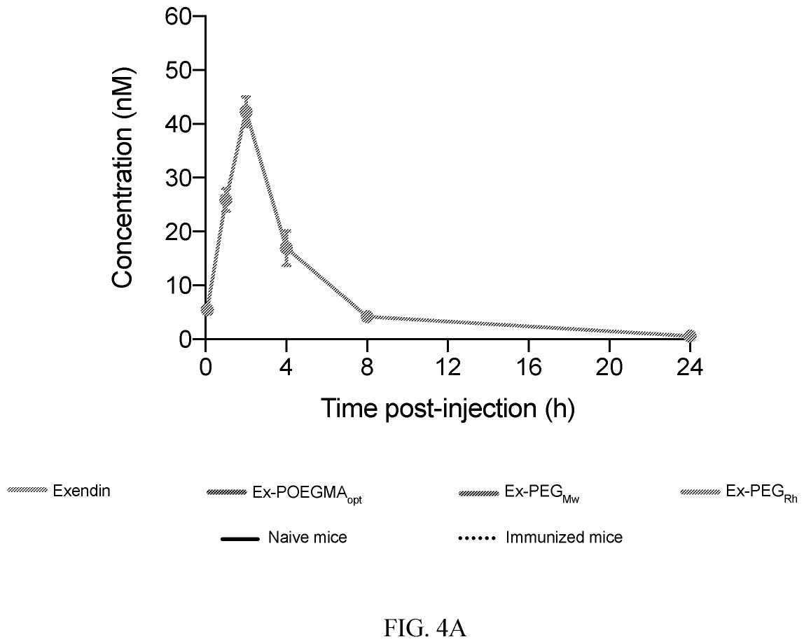

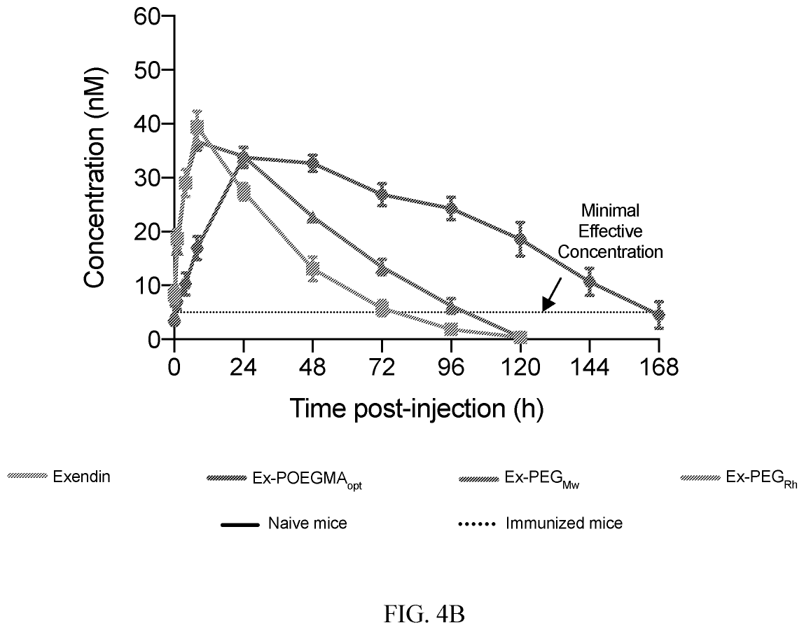

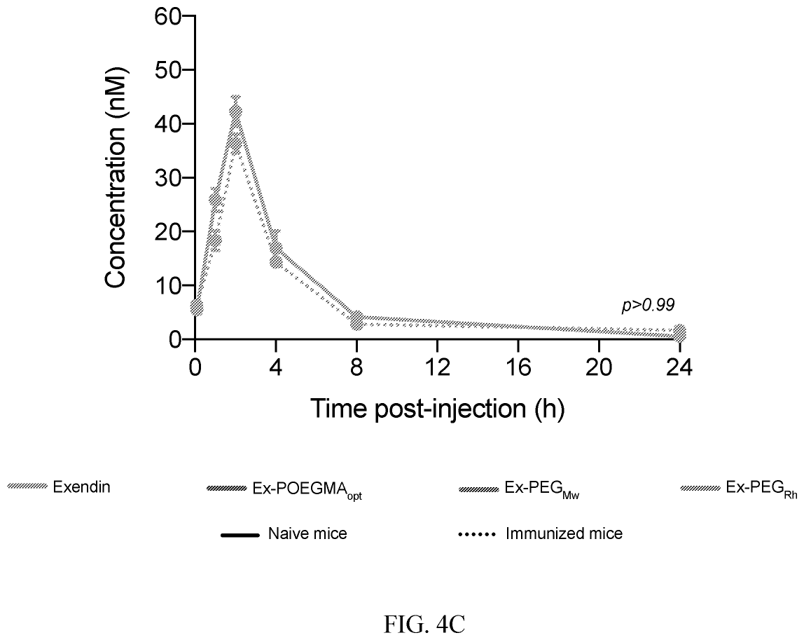

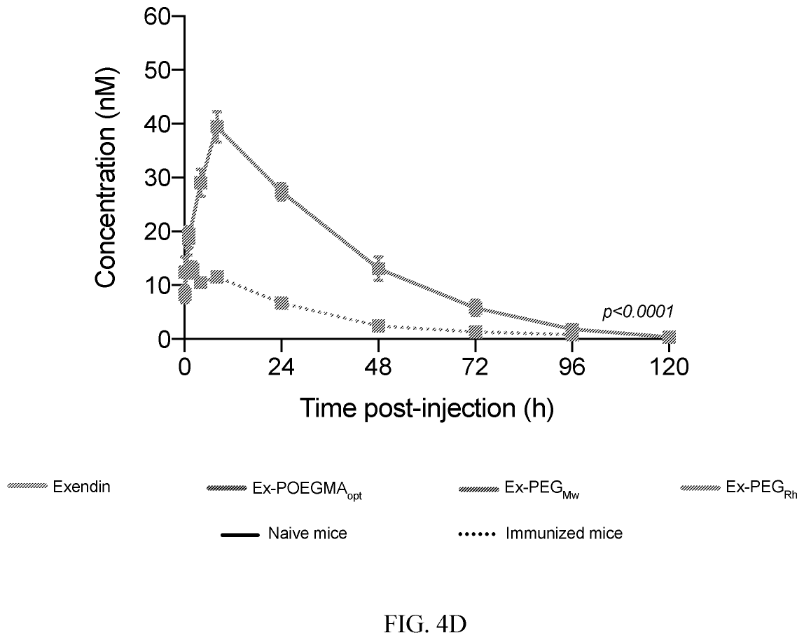

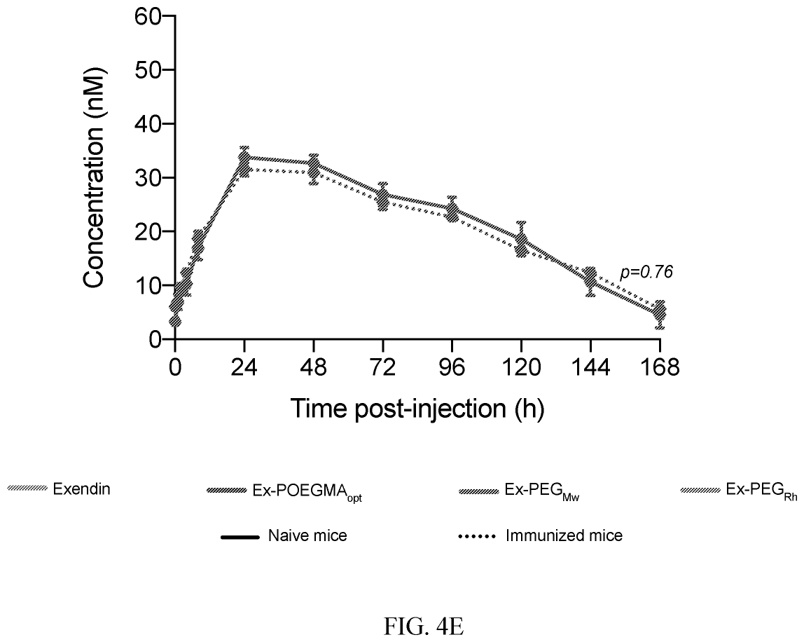

To better understand the differences in the short-term efficacy profiles, we next investigated the PK of Ex-POEGMAₒₚₜ. We fluorescently labeled exendin, Ex-POEGMAₒₚₜ, Ex-PEG_{Rh}, and Ex-PEG_{Mw}, followed by s.c. administration into naïve DIO mice (n=4). The PK parameters were determined from the drug's plasma concentration (FIG. 4A and FIG. 4B) and given in Table 3. The conjugates had much slower absorption kinetics than exendin, suggesting that the conjugates were primarily uptaken from the s.c. space into the blood through the lymphatic system due to their large size. Ex-POEGMAₒₚₜ had the slowest absorption (t_{1/2 absorption}=9.0±1.8 h) among the treatments, possibly due to its amphiphilic structure and sustained-release. Exendin had a fast elimination half-life (t_{1/2 elimination}=2.1±0.5 h), defined as the time needed to reach half-maximal blood concentration, due to its small size (FIG. 4A). The conjugates had much longer elimination half-lives due to their slower renal clearance (FIG. 4B). Ex-POEGMAₒₚₜ outperformed all conjugates in PK by prolonging exendin's circulation by ˜46-fold (t_{1/2 elimination}=97.3∓5.6 h) and providing the highest drug exposure (AUC) among the treatments. Ex-POEGMAₒₚₜ had a ˜2-fold longer elimination half-life than a 55.6 kDa soluble exendin-POEGMA conjugate with a matching M_{w}, Rₕ, and EC₅₀ (t_{1/20 elimination}=61.2±5.0 h), indicating the PK benefits provided by the sustained release. Ex-PEG_{Rh} (t_{1/2 elimination}=23.1±6.4 h) and Ex-PEG_{Mw} (t_{1/2 elimination}=34.9±9.1 h) prolonged exendin's circulation by ˜11- and ˜17-fold, respectively. Given that the soluble exendin-POEGMA conjugate had longer pharmacokinetics than both PEG conjugates, we concluded that M_{w} and Rₕ could not be a surrogate of PK when polymer architectures were significantly different. Lastly, we determined the minimal effective concentration for Ex-POEGMAₒₚₜ as 5.1 nM.

TABLE 3
Pharmacokinetic parameters of Ex-POEGMAₒₚₜ. Pharmacokinetic parameters were
calculated from the data given in FIG. 4. Data were fitted to a one-phase exponential decay curve
and analyzed via non-compartmental pharmacokinetic analysis using GraphPad Prism 8
software. Absorption phase pharmacokinetics of Ex-PEGRh in immunized mice were not
calculated (N/C) due to its low goodness-of-fit values. Data showed the mean ± standard error of
the mean (SEM)

| | | t_{1/2} | t_{1/2} | | | |
|---|---|---|---|---|---|---|
| | | absorption | elimination | | | AUC |
| | Treatment | (h⁻¹) | (h⁻¹) | tₘₐₓ(h)* | Cₘₐₓ(nM)* | (h × nM) |
| Naïve Mice | Exendin | 0.6 ± 0.1 | 2.1 ± 0.5 | 1.6 ± 0.2 | 31.3 ± 15 | 188 ± 18 |
| | Ex-PEG_{Rh} | 6.6 ± 0.5 | 23.1 ± 6.4 | 16.6 ± 2.2 | 34.6 ± 2.4 | 1585 ± 112 |
| | Ex-PEG_{Mw} | 6.6 ± 1.7 | 34.9 ± 9.1 | 19.5 ± 4.9 | 35.3 ± 4.5 | 2206 ± 84 |
| | Ex- | 9.0 ± 1.8 | 97.3 ± 5.6 | 32.1 ± 5.8 | 35.8 ± 2.6 | 3664 ± 167 |
| | POEGMAₒₚₜ | | | | | |
| Immunized | Exendin | 0.8 ± 0.1 | 1.9 ± 0.5 | 1.7 ± 0.3 | 36.9 ± 6.8 | 159 ± 18 |
| Mice | Ex-PEG_{Rh} | N/C | 11.4 ± 5.4 | N/C | N/C | 424 ± 35 |
| | Ex- | 12.1 ± 2.0 | 105.7 ± 12 | 42.6 ± 6.4 | 30.3 ± 4.0 | 3664 ± 167 |
| | POEGMAₒₚₜ | | | | | |

### Example 7

### Long-Term Efficacy

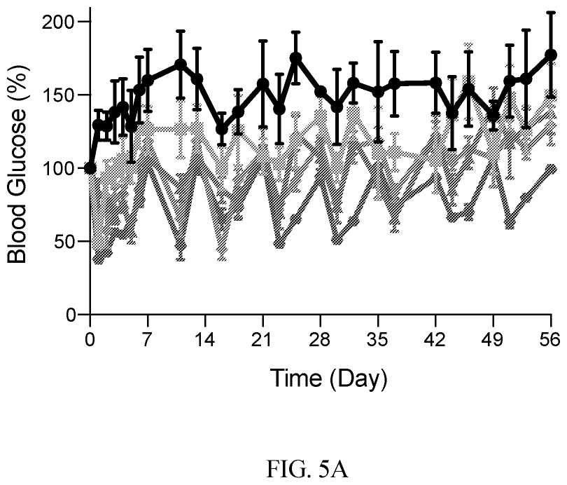

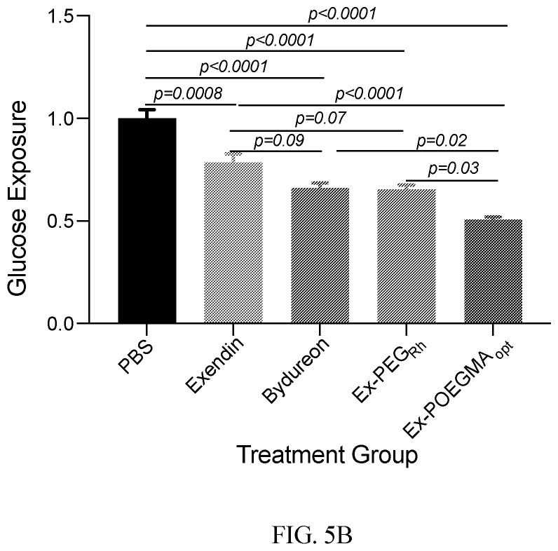

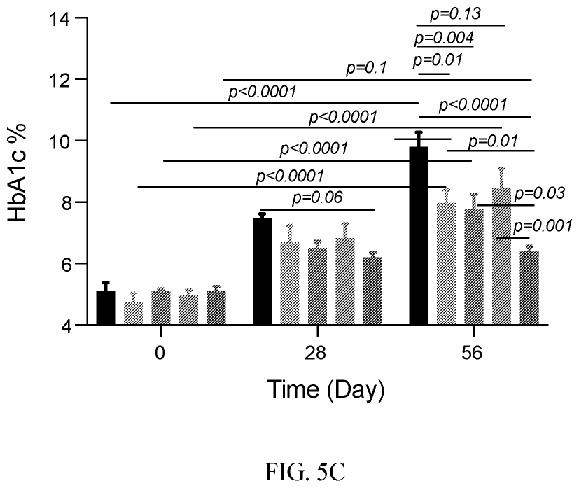

We next investigated the long-term efficacy of Ex-POEGMAₒₚₜ to understand better how the differences in PK and short-term efficacy translated into the long-term management of T2D. We hypothesized, without being bound to a particular theory, that Ex-POEGMAₒₚₜ should outperform other long-acting or sustained-release exendin formulations because of its superior fed blood glucose and glycemic control and longer PK. We tested this hypothesis by s.c. administering sterile and endotoxin-free Ex-POEGMAₒₚₜ, Ex-PEG_{Rh}, Bydureon, exendin, and PBS (-control) into naïve six-week-old male db/db mice (n=5) every week for eight weeks, followed by monitoring the blood glucose, changes in body weight, and glycated hemoglobin (HbA1c %) levels. HbA1c % is a measure of long-term T2D management because it is insensitive to daily blood glucose fluctuations and only changes as red blood cells (RBC) turn over every 40-60 days in rodents. All treatments resulted in lower fed blood glucose levels (FIG. 5A) and glucose exposure (FIG. 5B) than the control. Exendin only had a modest and short-lived effect due to its poor circulation. Long-term treatment with Bydureon resulted in sustained glucose control (FIG. 5A and FIG. 5B). Ex-PEG_{Rh} controlled fed blood glucose at varying degrees, with later injections providing inferior control, and resulted in a HbA1c % level that was not significantly different from PBS-treated mice (p>0.05). Ex-POEGMAopt consistently lowered fed blood glucose control after each injection and provided the lowest glucose exposure among treatments. Ex-POEGMAₒₚₜ outperformed all treatments by providing the lowest HbA1c %. Notably, HbA1c % was increased with age in all treatment groups except for Ex-POEGMAₒₚₜ. indicating superior T2D management. The sustained-release formulations Bydureon and Ex-POEGMAₒₚₜ resulted in a more moderate weight loss profile than Ex-PEG_{Rh}, consistent with the results after a single injection of the treatments.

### Example 8

### Immunogenicity

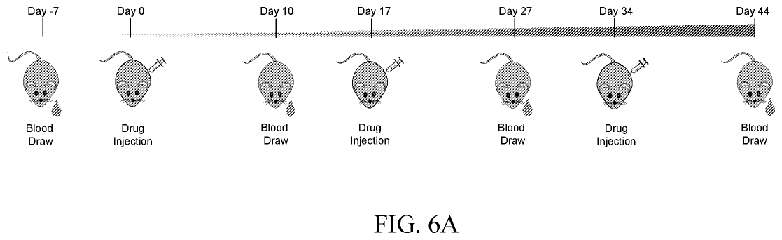

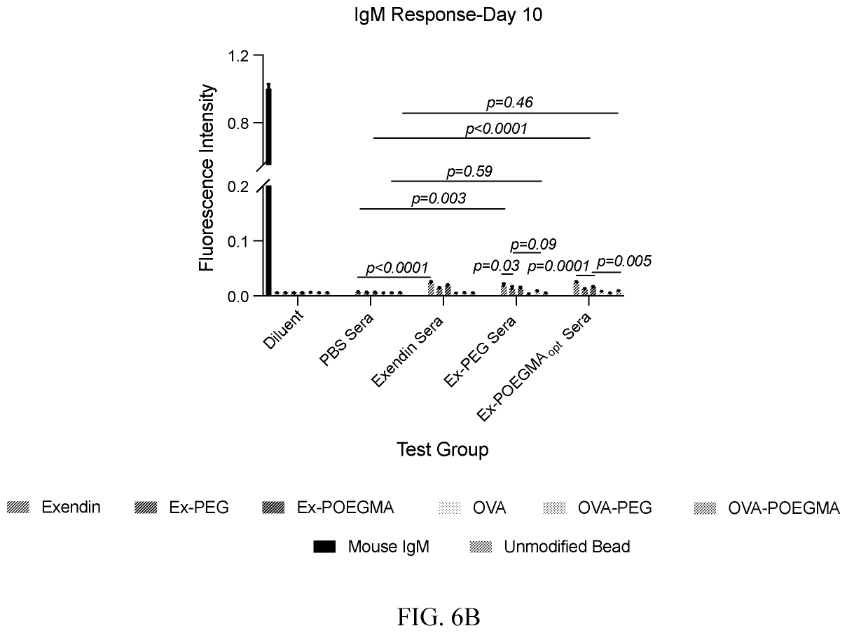

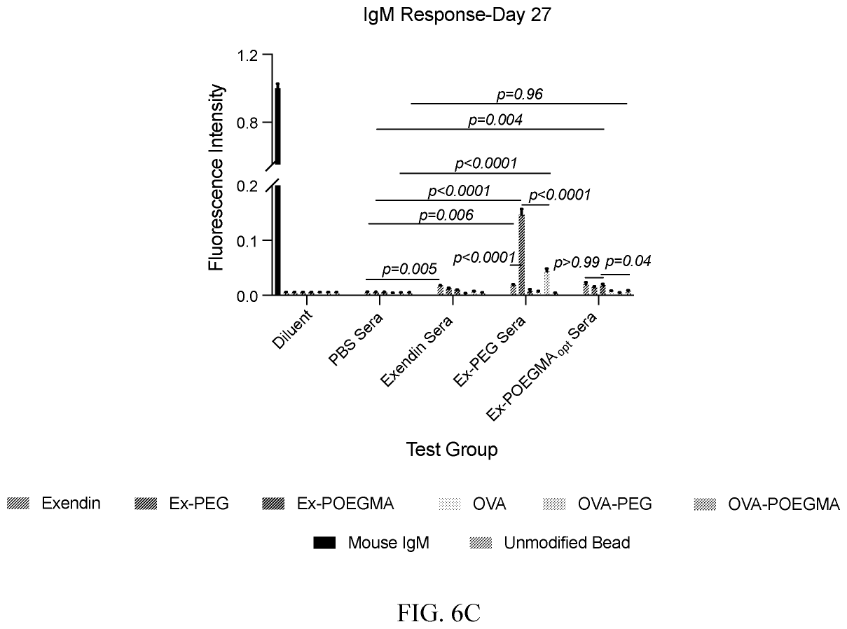

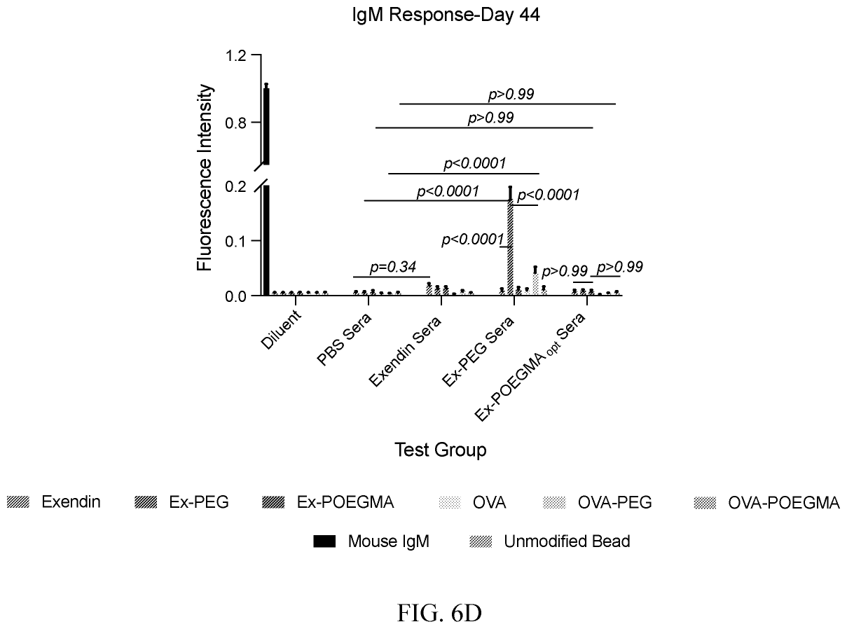

We next tested the immunogenicity of POEGMA in terms of induction of anti-drug antibodies (ADA) and compared it to PEG. PEG crosslinks the B-cell receptors (BCR) by repetitive structure, resulting in a T-cell independent B-cell immune response. This response is characterized by a persistent and predominantly Immunoglobulin (Ig) M-class ADA response in mouse. We hypothesized, without being bound to a particular theory, that POEGMA should not be immunogenic because of its hyperbranched structure. We tested this hypothesis by repeatedly s.c. administering sterile and endotoxin-free Ex-POEGMAₒₚₜ, Ex-PEG_{Mw}, exendin, and PBS (-control) into naïve DIO mice (n=10) (see dosing and blood sampling regimen in FIG. 6A). We assessed ADAs in terms of their titer, specificity (i.e., anti-conjugate, anti-exendin, anti-PEG, or anti-POEGMA), and subtype (i.e., IgM or IgG) using a Luminex multiplexed immunoassay (LMI). Briefly, the LMI used drug-conjugated, fluorescently barcoded magnetic beads to capture ADAs. We coupled exendin, exendin-PEG, and exendin-POEGMA onto separate sets of beads to determine ADAs induced by the treatments. Ovalbumin (OVA) versions of these beads—OVA, OVA-PEG, and OVA-POEGMA—were also coupled to separate sets of beads to use as a cross-reactivity control and determine PEG- and POEGMA-specific ADAs, respectively. Each drug-conjugated bead set had a different fluorescent barcode, allowing us to measure the signal detected by each of them when multiplexed. Mouse IgG- or IgM-coupled beads in diluent were used as positive controls, while the multiplexed beads in diluent served as a negative control.

Both free and conjugated exendin induced a transient IgM-class ADA response (FIG. 6B, FIG. 6C, and FIG. 6D). No IgG-class anti-exendin response was detected. Ex-PEG_{Mw} induced a significant and persistent anti-PEG ADA response (FIG. 6C and FIG. 6D). PEG specificity was indicated by the significant signal measured with both exendin-PEG and OVA-PEG beads. Anti-PEG ADAs were strictly IgM-class, and no maturation into IgG was observed. PEG-specific IgM response increased with the number of Ex-PEG_{Mw} injections. Notably, the induced PEG antibodies did not bind POEGMA conjugated bead sets, indicating that POEGMA eliminates PEG antigenicity. Strikingly, Ex-POEGMAₒₚₜ did not induce anti-POEGMA antibodies, indicating eliminated PEG immunogenicity.

We further tested the immunogenicity of POEGMA and compared it to PEG using highly immunogenic OVA as its conjugation partner. Briefly, OVA, OVA-PEG, and OVA-POEGMA were s.c. administrated into naïve C₅₇BL6/J mice (n=10) using PBS as a negative control by following the timeline given in FIG. 6A. The LMI described above was used to assess ADAs induced towards the treatments with minor changes in evaluating the results. Briefly, OVA, OVA-PEG, and OVA-POEGMA coupled beads were used to determine ADAs induced by the treatments, while exendin, exendin-PEG, and exendin-POEGMA coupled beads were used as a cross-reactivity control and determine PEG- and POEGMA-specific ADAs, respectively. Both free and conjugated OVA induced a significant IgG-class immune response. OVA-PEG conjugate induced lower titers of anti-OVA antibodies than unmodified OVA. This result was attributed to the steric hindrance imparted by the PEG, allowing the conjugate to expose fewer number of OVA epitopes to the immune system. The difference in anti-OVA antibody titers was not as drastic when POEGMA was used because it provides less steric hindrance than PEG to its conjugation partners due to its more compact architecture. PEG was highly immunogenic, indicated by the high titers of PEG-specific ADAs detected by OVA-PEG and exendin-PEG beads. Similar to when exendin used as a conjugation partner, PEG-specific ADAs remained restricted to the IgM class, possibly due to the lack of T-cell help. PEG-specific IgM titer was ˜60-fold higher with OVA-PEG than exendin-PEG, possibly due to the higher immunogenicity of OVA. In contrast, only a mild IgM-class anti-POEGMA response was detected after the first drug injection. The anti-POEGMA response was not detectable at later time points and did not mature into an IgG-type response, further iterating our conclusions on eliminated PEG immunogenicity with POEGMA.

Finally, we tested POEGMA's immunogenicity by administering OVA-POEGMA with Freund's adjuvant (FA) and compared it to OVA-PEG. FA significantly enhances the titer and affinity of the immune response induced towards co-administered immunogens by extending their presentation, stimulating the innate immune system, and facilitating T-cell help, allowing the immunogens to reveal their most immunogenic state. We hypothesized, without being bound to a particular theory, that POEGMA should at most induce a weak IgM-class immune response. We tested this hypothesis by s.c. administering PBS (-control), OVA, OVA-PEG, and OVA-POEGMA into naïve C₅₇BL/6J mice (n=10) as an emulsion of FA. The blood collections were made by following the timeline given in FIG. 6A, followed by plasma processing and the LMIs. Administering the treatments with FA increased anti-OVA and anti-PEG ADA titers by ˜2-fold, possibly because FA further stimulated antibody-producing plasma cell formation. Notably, no change in the specificity and subtype of the immune response developed towards the treatments was detected. Strikingly, POEGMA induced only a weak IgM response disappearing after the first injection, establishing the immunogenicity benefits of POEGMA over PEG.

### Example 9

### Effect of ADAs on Long-Term Efficacy

When immunogenicity and long-term efficacy data were evaluated together, they suggested that the inferior blood glucose control with the increasing number of injections and poor HbA1c % control by Ex-PEG_{Rh} could result from anti-PEG antibody interference. To test this hypothesis, we next investigated the ADAs' presence and function in the long-term treated mice sera collected on Day 66 using the LMI described earlier. Both exendin and Bydureon induced a mild anti-exendin IgM response. Notably, Ex-POEGMAₒₚₜ did not induce anti-POEGMA antibodies and resulted in a much weaker anti-exendin IgM response, possibly due to the non-immunogenic nature of POEGMA and the shielding of exendin's immunogenic epitopes by POEGMA Notably, anti-exendin IgM titers were higher in Ex-PEG_{Rh} treated mice sera, and anti-exendin IgG response was detected Ex-PEG_{Rh} also induced both IgM- and IgG-class PEG-specific antibody response. Given the absence of exendin- and PEG-specific IgGs in the immunogenicity experiment, the presence of higher-affinity class ADAs was attributed to the greater number of injections (3 vs. 8), more frequent dosing (17 vs. 7 days), and the longer time on the treatment (44 vs. 66 days) that are known to affect immune response.

We next investigated if the ADAs could neutralize the treatments. We analyzed long-term treated mice sera for neutralizing antibodies (NAbs) using a cell-based assay developed to test the GLP1R-binding by exendin variants with minor modifications. The GLP1R-binding by the treatments did not change when incubated with the mice sera, indicating that the ADAs did not neutralize the treatments. This result was consistent with the results from the clinical trials of exendin, where anti-exendin ADAs did not affect its PD. We also tested if PEG-specific ADAs neutralized Ex-PEG_{Mw} by incubating the conjugate with the plasma samples of the mice immunized with FA emulsion of OVA-PEG. Ex-PEG_{Mw} showed no change in activity, indicating that even high titers of anti-PEG antibodies did not have neutralizing activity.

We finally tested the effect of ADAs on the PK of the treatments. We hypothesized, without being bound to a particular theory, that the loss of efficacy in the Ex-PEG_{Mw} long-term treatment was due to the binding of the anti-PEG antibodies to the circulating drug, resulting in accelerated blood clearance and preventing it from showing efficacy. We also hypothesized, without being bound to a particular theory, that Ex-POEGMAₒₚₜ did not show a difference in PK among injections due to the lack of ADAs. To test these hypotheses, we s.c. administered sterile, endotoxin-free, and fluorescently-labeled exendin, Ex-PEG_{Rh}, and Ex-POEGMAₒₚₜ into DIO C₅₇BL/6J (n=4) and tracked their PK after the first (naïve mice) and fifth injection (immunized mice). Exendin did not show any difference in PK parameters between naïve and immunized mice (FIG. 4C; Table 2), indicating that anti-exendin antibodies were not PK-altering. Importantly, Ex-PEG_{Rh} showed significantly different PK profiles in naïve and immunized mice (FIG. 4D; Table 2). Its absorption was significantly lower in the immunized mice, suggesting local lymph nodes' involvement in its elimination. Ex-PEGRA's elimination half-life was ˜2-fold shorter in the immunized mice (t_{1/2 elimination}=11.4±5.4 h vs. 23.1±6.4 h), indicating the PK-altering nature of anti-PEG antibodies. Together, the ˜4-fold lower drug exposure in immunized mice (AUC=424±35 vs. 1585±112) indicated that anti-PEG immune response was responsible for the loss of efficacy in the long-term treatment with Ex-PEG_{Rh}. Notably, Ex-POEGMAₒₚₜ preserved its PK benefits after repeated administrations (FIG. 4E; Table 2), owing to its non-immunogenic structure.

### Example 10

### Histopathological Effects

We next investigated if Ex-POEGMAₒₚₜ had any histopathological effects and compared it to PEG, which induces significant vacuolization in major organs. We collected spleen, liver, kidney, pancreas, and thyroid tissues from the db/db mice (n=3) used in the long-term study, followed by processing for histological analyses and hematoxylin and eosin (H&E) staining. No compound induced changes were noted within the splenic or thyroid tissues. Centrilobular macrovesicular steatosis, a characteristic of T2D, was observed in hepatic lobules in all groups, indicated by fat-filled vacuoles. The lesions varied in severity and incidence across the groups, with Ex-POEGMAₒₚₜ showing a trend towards displaying the mildest steatosis among the treatments, supporting the long-term efficacy results. Exendin and Bydureon did not have any histopathological effects on the kidney, while significant vacuolization was noted in the Ex-PEG_{Rh} group. The vacuolar lesions were prominent primarily within renal tubular epithelial cells of proximal tubules in the outer cortical region. A minimal compound-induced vacuolar change was also observed in pancreatic acinar cells of all Ex-PEG_{Rh}-treated mice, possibly because of GLP1R targeting by exendin. Importantly, Ex-POEGMAₒₚₜ induced no histopathological changes in the kidney and pancreas.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method of treating a disease in a subject in need thereof, the method comprising administering to the subject an effective amount of a composition, the composition comprising a plurality of conjugates, the conjugate comprising
a biologically active agent; and
a copolymer of poly[oligo(ethylene glycol) ether methacrylate] (POEGMA) conjugated to the biologically active agent, the copolymer of POEGMA comprising recurring units of formula (1)

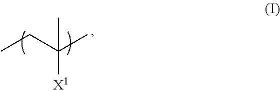

wherein
X¹ is of formula (II)

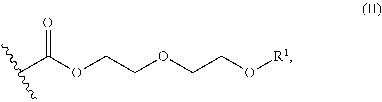

or formula (III)

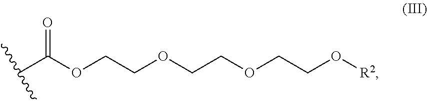

wherein R¹ and R² are each independently hydrogen, alkyl, ester, C₁-C₄ alkylenyl-NH₂, amide, carboxyl, or C₁-C₄ alkylenyl-OH, wherein the copolymer of POEGMA comprises about 1 molar % to about 99 molar % of recurring units with formula (II), about 1 molar % to about 99 molar % of recurring units with formula (III), and a weight average molecular weight of about 2 kDa to about 500 kDa, and

wherein the conjugate has a transition temperature (Tₜ) of about 25° C. to about 37° C. at a concentration of about 1 uM to about 1 M, the plurality of conjugates self-assemble into an aggregate above the Tₜ of the conjugate, and the composition does not induce a histopathological change in the subject.

Clause 2. The method of clause 1, wherein the conjugate has a reduced immune response relative to a polyethylene glycol (PEG)-biologically active agent conjugate.

Clause 3. The method of clause 1 or 2, wherein the conjugate does not induce an anti-POEGMA antibody response.

Clause 4. The method of any one of clauses 1-3, wherein the conjugate does not induce an anti-POEGMA IgG response, an anti-POEGMA IgM response, or both.

Clause 5. The method of any one of clauses 1-4, wherein the conjugate is not reactive with pre-existing anti-PEG antibodies in the subject.

Clause 6. The method of any one of clauses 1-5, wherein the biologically active agent is conjugated to the copolymer of POEGMA via a triazole.

Clause 7. The method of any one of clauses 1-6, wherein the biologically active agent is conjugated to the backbone of the copolymer of POEGMA.

Clause 8. The method of any one of clauses 1-7, wherein the biologically active agent comprises a nucleotide, a polynucleotide, a protein, a peptide, a polypeptide, a carbohydrate, a lipid, a small molecule drug, or a combination thereof.

Clause 9. The method of any one of clauses 1-8, wherein the biologically active agent comprises a protein, a peptide, or a polypeptide.

Clause 10. The method of any one of clauses 1-9, wherein the biologically active agent is exendin.

Clause 11. The method of any one of clauses 1-10, wherein R¹ and R² are each independently methyl.

Clause 12. The method of any one of clauses 1-11, wherein the copolymer of POEGMA comprises about 40 molar % to about 75 molar % of recurring units with formula (II) and about 25 molar % to about 60 molar % of recurring units with formula (III), the copolymer of POEGMA has a weight average molecular weight of about 20 kDa to about 60 kDa; and the conjugate has a Tₜ of about 28° C. to about 32° C. at a concentration of about 500 μM.

Clause 13. The method of any one of clauses 1-12, wherein the composition is administered at a temperature below the conjugate's Tₜ to an area of the subject that has a temperature above the conjugate's Tₜ.

Clause 14. The method of any one of clauses 1-13, wherein the composition is administered subcutaneously, intradermally, intramuscularly, or intraperitoneally.

Clause 15. The method of any one of clauses 1-14, wherein the disease is a cancer, a metabolic disease, an autoimmune disease, a cardiovascular disease, or an orthopedic disorder.

Clause 16. The method of any one of clauses 1-15, wherein the disease is a metabolic disease selected from the group consisting of obesity, type 2 diabetes mellitus, pancreatitis, dyslipidemia, nonalcoholic fatty liver disease (NAFLD), nonalcoholic steatohepatitis (NASH), insulin resistance, hyperinsulinemia, glucose intolerance, hyperglycemia, and a combination thereof.

Clause 17. The method of clause 16, wherein administration of the composition results in the subject having at least one of decreased blood glucose, decreased body fat, increased insulin production, decreased hemoglobin A1c values, decreased circulating fatty acids, decreased liver fat content, decreased liver inflammation, and decreased liver fibrosis compared to the subject not receiving the administration of the composition.

Clause 18. The method of any one of clauses 1-17, wherein the subject has decreased blood glucose for at least 6 days after a single administration of the composition compared to the subject not receiving the administration of the composition.

What is claimed is:

1. A method of treating a disease in a subject in need thereof, the method comprising administering to the subject an effective amount of a composition, the composition comprising a plurality of conjugates, the conjugate comprising
a biologically active agent; and
a copolymer of poly[oligo(ethylene glycol) ether methacrylate] (POEGMA) conjugated to the biologically active agent, the copolymer of POEGMA comprising recurring units of formula (I) 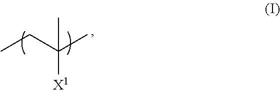
wherein
X¹ is of formula (II) 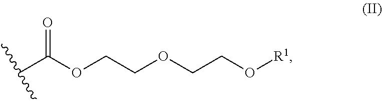
or
formula (III) 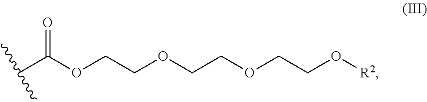
wherein R¹ and R² are each independently hydrogen, alkyl, ester, C₁-C₄ alkylenyl-NH₂, amide, carboxyl, or C₁-C₄ alkylenyl-OH, wherein the copolymer of POEGMA comprises about 1 molar % to about 99 molar % of recurring units with formula (II), about 1 molar % to about 99 molar % of recurring units with formula (III), and a weight average molecular weight of about 2 kDa to about 500 kDa, and
wherein the conjugate has a transition temperature (Tₜ) of about 25° C. to about 37° C. at a concentration of about 1 uM to about 1 M, the plurality of conjugates self-assemble into an aggregate above the Tₜ of the conjugate, and the composition does not induce a histopathological change in the subject.

2. The method of claim 1, wherein the conjugate has a reduced immune response relative to a polyethylene glycol (PEG)-biologically active agent conjugate.

3. The method of claim 1, wherein the conjugate does not induce an anti-POEGMA antibody response.

4. The method of claim 1, wherein the conjugate does not induce an anti-POEGMA IgG response, an anti-POEGMA IgM response, or both.

5. The method of claim 1, wherein the conjugate is not reactive with pre-existing anti-PEG antibodies in the subject.

6. The method of claim 1, wherein the biologically active agent is conjugated to the copolymer of POEGMA via a triazole.

7. The method of claim 1, wherein the biologically active agent is conjugated to the backbone of the copolymer of POEGMA.

8. The method of claim 1, wherein the biologically active agent comprises a nucleotide, a polynucleotide, a protein, a peptide, a polypeptide, a carbohydrate, a lipid, a small molecule drug, or a combination thereof.

9. The method of claim 1, wherein the biologically active agent comprises a protein, a peptide, or a polypeptide.

10. The method of claim 1, wherein the biologically active agent is exendin.

11. The method of claim 1, wherein R¹ and R² are each independently methyl.

12. The method of claim 1, wherein:
the copolymer of POEGMA comprises about 40 molar % to about 75 molar % of recurring units with formula (II) and about 25 molar % to about 60 molar % of recurring units with formula (III);
the copolymer of POEGMA has a weight average molecular weight of about 20 kDa to about 60 kDa; and
the conjugate has a Tₜ of about 28° C. to about 32° C. at a concentration of about 500 μM.

13. The method of claim 1, wherein the composition is administered at a temperature below the conjugate's Tₜ to an area of the subject that has a temperature above the conjugate's Tₜ.

14. The method of claim 1, wherein the composition is administered subcutaneously, intradermally, intramuscularly, or intraperitoneally.

15. The method of claim 1, wherein the disease is a cancer, a metabolic disease, an autoimmune disease, a cardiovascular disease, or an orthopedic disorder.

16. The method of claim 15, wherein the disease is a metabolic disease selected from the group consisting of obesity, type 2 diabetes mellitus, pancreatitis, dyslipidemia, nonalcoholic fatty liver disease (NAFLD), nonalcoholic steatohepatitis (NASH), insulin resistance, hyperinsulinemia, glucose intolerance, hyperglycemia, and a combination thereof.

17. The method of claim 16, wherein administration of the composition results in the subject having at least one of decreased blood glucose, decreased body fat, increased insulin production, decreased hemoglobin A1c values, decreased circulating fatty acids, decreased liver fat content, decreased liver inflammation, and decreased liver fibrosis compared to the subject not receiving the administration of the composition.

18. The method of claim 1, wherein the subject has decreased blood glucose for at least 6 days after a single administration of the composition compared to the subject not receiving the administration of the composition.
